(19) United States
(12) Patent Application Publication
(10) Pub. No.: US 2024/0361331 A1
(43) Pub. Date: Oct. 31, 2024

(54) SYSTEMS AND METHODS FOR ANALYSIS OF PEPTIDE PHOTODISSOCIATION FOR SINGLEMOLECULE PROTEIN SEQUENCING

(71) Applicant: Brown University, Providence, RI (US)

(72) Inventors: Derek M. Stein, Providence, RI (US); Jacob Sage Vietorisz, Stamford, CT (US); Nicholas Drachman, Providence, RI (US)

(73) Assignee: Brown University, Providence, RI (US)

(21) Appl. No.: 18/681,908

(22) PCT Filed: Aug. 18, 2022
(86) PCT No.: PCT/US2022/040725

§ 371 Date: Feb. 7, 2024

Related U.S. Application Data
(60) Provisional application No. 63/235,601, filed on Aug. 20, 2021.
(60) Provisional application No. 63/341,992, filed on May 13, 2022.

(51) Int. Cl. G01N 33/68; B82Y 15/00; G01N 33/487; H01J 49/00

(52) U.S. Cl. CPC G01N 33/6848; G01N 33/48721; H01J 49/0059; B82Y 15/00

(57) ABSTRACT

The present disclosure generally relates to systems and methods for analysis of peptide photodissociation for single-molecule protein sequencing. In one aspect, systems and methods are directed to allowing one to fragment a single protein molecule in aqueous solution so that its composition and sequence of amino acids can be measured by mass spectrometry. This may be useful for single-molecule protein sequencing technology.

### RELATED APPLICATION

This application claims the benefit of U.S. Provisional Patent Application Ser. No. 63/341,992, filed May 13, 2022, entitled “Systems and Methods for Analysis of Peptide Photodissociation for Single-Molecule Protein Sequencing”, and U.S. Provisional Patent Application Ser. No. 63/235,601, filed Aug. 20, 2021, entitled “Systems and Methods for Analysis of Peptide Photodissociation for Single-Molecule Protein Sequencing.” each of which is incorporated herein by reference in its entirety.

### FIELD

The present disclosure generally relates to systems and methods for analysis of peptide photodissociation for single-molecule protein sequencing.

### BACKGROUND

Peptide sequencing is an essential tool of proteomics that is widely used for identifying proteins and mapping the protein content of cells. An ability to sequence a single copy of a protein would significantly improve analyses of single cells and enable the study of low-abundance proteins and proteoforms, which can be present in fewer than 10 copies per cell. A single-molecule technology is required because proteins cannot be biochemically amplified like nucleic acids. Furthermore, post-translational modifications and proteoforms from alternative mRNA splicings cannot be inferred from DNA or RNA sequences. New diagnostic applications and drug therapies could also result from single-molecule protein sequencing. There is growing interest in developing techniques for sequencing single proteins based on fluorescence tagging, N-terminus probing, and nanopores.

Mass spectrometry (MS) is the current workhorse of peptide sequencing. However, the technique typically requires 10⁷ or more copies of a protein to reach the limit of detection. The MS ion source typically limits the sensitivity more than any other component. In electrospray ionization, charged analyte-containing droplets collide with a background gas in order to release analyte ions into the gas phase, and the released ions may be then transferred through the instrument to the detector; the combined efficiency of those processes is very low. Thus, improvements are needed.

### SUMMARY

The present disclosure generally relates to systems and methods for analysis of peptide photodissociation for single-molecule protein sequencing. The subject matter of the present disclosure involves, in some cases, interrelated products, alternative solutions to a particular problem, and/or a plurality of different uses of one or more systems and/or articles.

In one aspect, systems and methods are directed to allowing one to fragment a single protein molecule in aqueous solution so that its composition and sequence of amino acids can be measured by mass spectrometry. This may be useful for single-molecule protein sequencing technology. Single-molecule protein sequencing is the next frontier in biomolecular diagnostics, and its development may help revolutionize the field of biology and disease diagnostics.

In some cases, these may be implemented using hardware that could be commercialized as add-on components for mass spectrometry systems. In addition, certain embodiments are generally directed to a single-molecule protein sequencing instrument, which could be used in settings such as biomedical research and in the clinic.

Certain methods and systems may allow proteins to be fragmented in aqueous solution, rather than in the gas phase. In some embodiments, the peptide bonds linking amino acids to the parent peptide may be selectively cleaved. In some cases, amino acids may be liberated intact for analysis by mass spectrometry. These methods may be compatible with a single-molecule protein sequencing strategy, e.g., as discussed in Int. Pat. Apl. Pub. No. PCT/US2021/028954, filed Apr. 23, 2021 and U.S. Pat. Apl. Ser. No. 63/179,046, filed Apr. 23, 2021, each incorporated herein by reference.

In one set of embodiments, the method comprises using light to fragment a protein into single molecules in a mass spectrometer.

According to another set of embodiments, the method comprises arranging a protein into a substantially linear configuration in a nanotip; fragmenting the protein into amino acids by applying laser light to the protein; emitting the amino acids from the nanotip; and detecting the amino acids emitted from the nanotip.

The method, in another set of embodiments, comprises applying light at a wavelength of greater than or equal to 150 nm and less than or equal to 213 nm to a protein to cleave fragments from the protein; and sequencing the fragments using mass spectrometry.

According to yet another set of embodiments, the method comprises applying light at a wavelength of greater than or equal to 150 nm and less than or equal to of 222 nm to a protein to cleave fragments from the protein; and sequencing the fragments using mass spectrometry.

According to yet another set of embodiments, the method comprises applying laser light to a protein to cleave amino acids from the protein; and sequencing the amino acids using mass spectrometry.

According to yet another set of embodiments, the method comprises applying light of a wavelength of greater than or equal to 150 nm and less than or equal to 213 nm to a peptide to cleave fragments from the peptide; passing at least 50% of the fragments through a magnetic mass filter; and directing the fragments to a detector.

One aspect of the disclosure is generally directed to a method of sequencing a protein. According to one set of embodiments, the method of sequencing a protein comprises fragmenting a protein to produce fragments by applying light at a wavelength of greater than or equal to 150 nm and less than or equal to 213 nm to the protein; passing the fragments through a magnetic mass filter; directing the fragments to an array of detectors; and determining a sequence of the protein by determining the fragments with the array of detectors.

According to another set of embodiments, the method of sequencing a protein comprises passing a fluid comprising a protein into a capillary defining an opening; applying light at a wavelength of greater than or equal to 150 nm and less than or equal to 213 nm to the protein proximate the opening to produce fragments; passing the fragments directly into an environment having a pressure of no more than 100 mPa; passing the fragments through a magnetic mass filter; directing the fragments to an array of detectors; and determining a sequence of the protein by determining the fragments with the array of detectors.

One aspect of the present disclosure is generally directed to a mass spectrometer. According to one set of embodiments, the mass spectrometer comprises a nanotip that allows a protein to be arranged into a linear configuration; and a laser positioned to direct light to dissociate the protein into fragments.

According to another set of embodiments, the mass spectrometer comprises an ion source comprising a capillary; a light source directed towards the ion source, wherein the light source is able to produce light at a wavelength of greater than or equal to 150 nm and less than or equal to 213 nm; a magnetic mass filter downstream of the ion source; and an array of detectors downstream of the magnetic mass filter.

According to yet another set of embodiments, the mass spectrometer comprises an ion source comprising a capillary; a laser positioned to direct light at the capillary; and a detector position downstream of the ion source.

According to yet another set of embodiments, the mass spectrometer comprises an ion source comprising a capillary and an electrode proximate the capillary, wherein the capillary comprises an opening having a cross-sectional dimension of less than 125 nm; a magnetic mass filter downstream of the ion source; an array of detectors downstream of the magnetic mass filter; and a light source directed towards the ion source, wherein the light source is able to produce light at a wavelength of greater than or equal to 150 nm and less than or equal to 213 nm.

In another aspect, the present disclosure encompasses methods of making one or more of the embodiments described herein. In still another aspect, the present disclosure encompasses methods of using one or more of the embodiments described herein.

Other advantages and novel features of the present disclosure will become apparent from the following detailed description of various non-limiting embodiments of the disclosure when considered in conjunction with the accompanying figures.

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure.

### BRIEF DESCRIPTION OF THE DRAWINGS

FIG. 1A shows a schematic of single-molecule protein sequencing by nanopore mass spectrometry.

FIG. 1B shows an elongated peptide chain photofragmenting near the tip of the nanocapillary ion source.

FIG. 2 shows the structure of a dipeptide, the structures of common photofragmentation products, and the approximate frequency with which laser light in different wavelength regimes induces particular transformations in vacuum.

FIG. 3A shows a calculated heating profile in a nanocapillary under steady irradiation by 10.6 μm, 193 nm, and 222 nm light.

FIG. 3B shows dependence of the maximum temperature increase in a nanocapillary on the incident laser power density for 10.6 micrometers (triangles), 193 nm (squares), and 222 nm (dots) light. Symbols show results of finite element calculations, and curves are linear fits to the data.

FIG. 4A shows cumulative probability of peptide bond dissociation, obtained from Eq. 2 for exposure to 193 nm laser light of different intensities, as indicated.

FIG. 4B shows the probability of amino acids not decomposing as a function of exposure time to 193 nm laser light with ρ=10,000 Wm⁻², calculated according to Eq. 3.

FIG. 4C shows the probability of selective amino acid liberation (i.e., fragmenting the two peptide bonds joining an amino acid to a peptide without damaging the amino acid) as a function of exposure time to 193 nm laser light with ρ=10,000 Wm⁻².

FIG. 5A is a schematic of conventional electrospray ionization highlighting the background gas that stimulates evaporation of solvent from droplets and the transfer capillary where significant ion loss occurs.

FIG. 5B is a schematic of a nanopore ion source showing the liquid-filled nanocapillary tip, the extractor electrode, and the extraction voltage Vₑ applied between them. Inset shows an SEM image of the tip of a pulled quartz nanocapillary with a tip inner diameter of 30 nm.

FIG. 5C is a schematic of the mass spectrometer used in Example 2. Ion optics comprising an extractor electrode and an einzel lens extract ions from the liquid meniscus at the ion source and focus them through a quadrupole mass filter and an electrostatic ion bender. The transmitted ions strike a channel electron multiplier detector which is sensitive to single ions.

FIG. 5D shows a mass spectrum of 100 mM arginine in aqueous solution obtained with a 41 nm inner diameter nanopore ion source in the quadrupole mass spectrometer described herein.

FIG. 6A shows a mass spectrum of 100 mM arginine solution in H2O using nanopore ion sources with 3 different inner tip diameters (20 nm, 125 nm, and 300 nm).

FIG. 6B shows a gallery of 16 amino acid mass spectra, ordered from top left to bottom right by mass. All experiments were carried out using nanocapillaries with 20-60 nm inner tip diameters.

FIG. 6C shows an overlaid mass spectra of glutathione and two of its PTM variants, s-nitrosoglutathione and s-acetylglutathione.

FIG. 7A shows an experimental setup to measure the ion transmission efficiency of the nanopore ion source. A voltage V_{T} is applied to the nanopore ion source by a source-measure unit to produce the tip current I_{T}. The emitted ions are focused by the ion optics and strike the Faraday cup where the collected current I_{C} is measured by a current-to-voltage preamplifier. The experiment is carried out inside a vacuum chamber at pressures of around 10⁻⁷ torr. The ion transmission efficiency is the ratio of collected current to tip current I_{C}/I_{T}.

FIG. 7B shows a plot of the ion transmission efficiency measured over several minutes from a nanopore ion source using 100 mM NaI in water. The inset shows I_{T} and I_{C} plotted over the same time period.

FIG. 7C shows the experimental setup used to measure the relative fraction of ion current, I_{Ion}, and droplet current, I_{Drop}. A magnetic sector (diameter=6 cm, B-field strength=0.54 T) is placed past the ion optics to deflect emitted particles by their mass-to-charge ratio. I_{Ion} is collected by a wide Faraday plate and I_{Drop} is collected by the Faraday cup.

FIG. 7D shows I_{Ion}, I_{Drop}, and the ionic fraction of the total measured current for a 2 minute-long measurement performed using a 28 nm tip filled with a 100 mM aqueous solution of NaI

FIG. 8 shows the probability that an amino acid ion dressed with a hydration shell collides with a gas molecule, based on the kinetic theory of gases. The curve is the cumulative probability that an emitted amino acid with a small hydration shell (radius=7 Å) will collide with an evaporated water molecule or background N₂ molecule as a function of distance from the meniscus, r. The line shows the calculated maximum possible water vapor density as a function of the distance from the meniscus. The vapor pressure on the vacuum side of the meniscus was conservatively assumed to be 8.75 torr, half the equilibrium vapor pressure of water at room temperature. The inset shows a schematic representation of the distribution of evaporating water molecules near the meniscus.

FIG. 9A is a diagram showing water evaporating from a hemispherical meniscus, the tip radius r₀, and the distance r.

FIG. 9B shows a plot of Cₚ(r) (solid line) and the separate contributions from water molecules (dashed line) and background gas (dash-dot line) to the total probability of undergoing at least one collision. The dashed curve shows the contribution from evaporated water molecules, the dashed and dotted curve shows the contribution from the background gas, and the solid curve shows the total collision probability obtained by summing the contributions from the water and background gas. The calculation used n_{b}=2.25×10¹⁵ m⁻³, n_{w0}=6.44×10²³ m⁻³, aw=1.325 Å, a_{b}=1.82 Å, aᵢ=7 Å, and r₀=30 nm. The plots extend to r=0.5 m, the total distance from the meniscus to the detector.

FIG. 10 shows a plot of the measured fluidic conductance fit to the theoretical prediction using a semi-infinite truncated cone model of the nanocapillary, where the half-apex angle θ was used as a fitting parameter.

FIG. 11A shows simulation results of deflection angle versus m/z for singly charged ions. The region between the dashed lines represents the position and extent of the Faraday plate detector, so ions with 70<m/Z<325 should strike the Faraday plate.

FIG. 11B shows simulation results of deflection angle versus droplet radius for droplets of water charged to the Rayleigh limit. The region below the dashed line represents the position and extent of the Faraday cup aperture, such that fully charged droplets larger than 15 nm should strike the Faraday cup.

FIG. 11C shows the selection of simulated trajectories for five singly charged ions of mass between 60 and 460 amu, the arrow points in the direction of increasing mass, and the circle represents the magnetic sector.

FIG. 11D shows the selection of simulated trajectories for 5 droplets charged to the Rayleigh limit with radius between 5 and 25 nm, the arrow points in direction of increasing radius.

FIG. 12 shows a schematic of a multiplex mass spectrometer comprising a plurality of nanopore ion sources, in accordance with some embodiments.

### DETAILED DESCRIPTION

The present disclosure generally relates to systems and methods for analysis of peptide photodissociation for single-molecule protein sequencing. In one aspect, systems and methods are directed to allowing one to fragment a single protein molecule in aqueous solution so that its composition and sequence of amino acids can be measured by mass spectrometry. This may be useful for single-molecule protein sequencing technology.

Certain aspects of the disclosure are related to a mass spectrometer and interrelated methods that allow for single molecule fragmentation and sequencing of a species of interest (e.g., a polymer, a biopolymer, etc.). In some cases, the species of interest is a protein and methods related to single-molecule protein sequencing are disclosed herein. While some embodiments of the present disclosure relate to methods for analyzing and/or sequencing peptides and proteins, it should be understood that the present disclosure is not so limited, and that in other embodiments, the method may be employed for analyzing any of a variety of molecules and/or ions, including, but not limited to, salt ions, macromolecules, etc.

In some embodiments, a mass spectrometer comprising a light source configured to fragment a species of interest (e.g., protein) into individual components (e.g., amino acids) is disclosed herein. In some such embodiments, the mass spectrometer is a nanopore mass spectrometer that comprises an ion source capable of ionizing the species of interest into vacuum, as well as other associated components, including, but not limited to, a vacuum, a magnetic mass filter, and one or more detectors, as described in more detail below. The combinations of a light source, an ion source, etc., may advantageously allow for fragmentation of a species of interest (e.g., a biopolymer such as a protein) into base fragments (e.g., a monomer such as an amino acid) and sequencing thereof. The ion source, magnetic mass filter, one or more detectors, may have any properties, configurations, and/or arrangements are described in more detail below.

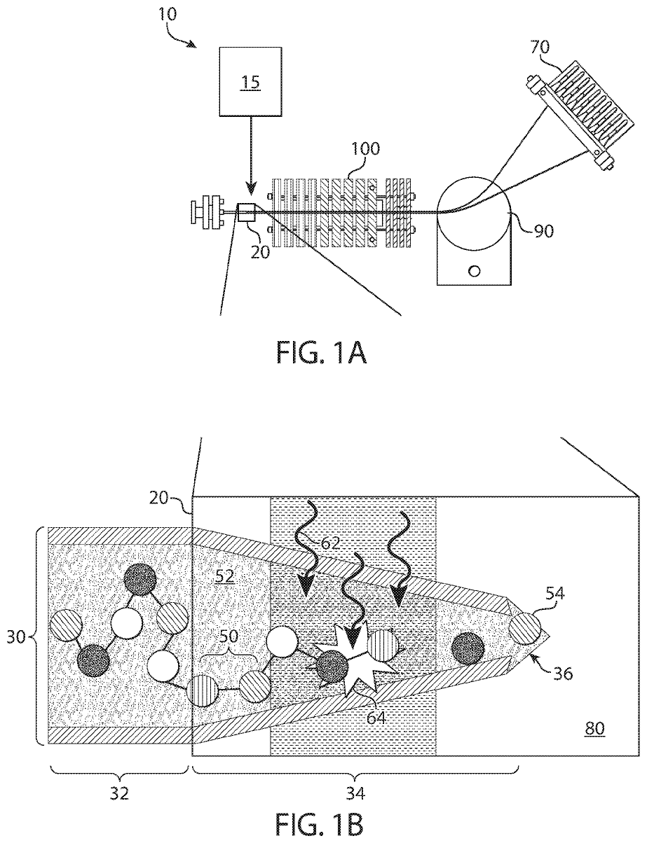

In one set of embodiments, the light source (e.g., a laser) may be positioned adjacent the ion source comprising a capillary, e.g., such that the light source is directed toward the ion source or a portion thereof. A non-limiting example of the mass spectrometer comprising such a light source is shown in FIGS. 1A-1B. As shown, the mass spectrometer 10 comprises light source 15 (e.g., a laser such as an UV laser) directed toward an ion source 20 comprising a capillary 30 filled with a fluid 52 containing a species of interest 50 (e.g., a protein or a peptide). The fluid may comprise any appropriate solvents described elsewhere herein, e.g., such as water, formamide, high volatility solvents, aqueous solutions, etc. In some cases, the light source may be directed toward the capillary tip (e.g., nanotip) of the ion source. In some embodiments, the light source may be directed toward the fluid containing the species of interest (e.g., protein or peptide) disposed within the capillary tip (e.g., the nanotip). For example, as shown in FIGS. 1A-1B, the light source 15 may be directed toward the fluid 52 containing the species of interest 50 within the capillary tip 34 (e.g., nanotip) of the ion source 20.

According to some embodiments, the light source, by being directed toward the fluid containing the species of interest, may be configured to fragment the species of interest (e.g., protein) in the fluid within the capillary into fragmented individual components or single molecules (e.g., individual amino acids). The fragmented individual components or single molecules, in accordance with some embodiments, may be subsequently ionized from an opening of the capillary tip (e.g., nanotip) into vacuum. In some embodiments, the vacuum chamber houses the nanotip. For example, as shown in FIG. 1B, the light source 15, by being directed toward the fluid 52 containing the species of interest 50, may be configured to fragment the species of interest 50 (e.g., protein or peptide) in the fluid 52 within the capillary tip 34 (e.g., nanotip) into fragmented individual components or single molecules 54 (e.g., amino acids). For example, light 62 (e.g., UV photons) emitted by light source 15 may lead to fragmentation 64 of the portion of species of interest 50 exposed to light 62. The fragmented individual components or single molecules 54 may be subsequently ionized from an opening 36 of the capillary tip 36 into a vacuum 80 housing the capillary tip 34. Methods directed to fragmenting the species of interest are described in more detail below.

In some embodiments, methods related to fragmentating a species of interest (e.g., a biopolymer such as protein) into individual components (e.g., individual amino acids) and sequencing thereof are disclosed herein. It should be noted such a method may be particularly beneficial for sequencing of a biopolymer (e.g., protein), where single-molecule precision is desired.

In some embodiments, the method comprises a step of arranging a species of interest (e.g., a protein) into a substantially linear configuration in the nanotip of the capillary. In some cases, the nanotip of the capillary may be sized such that it drives the species of interest to arrange itself into a linear configuration. As shown in FIGS. 1A-1B, the capillary tip 34 may be sized (e.g., by having a cross-sectional dimension (e.g., a maximum cross-sectional dimension) of less than 200 nm, less than 150 nm, less than 120 nm, less than 100 nm, less than 80 nm, less than 65 nm, less than 60 nm, less than 50 nm, less than 30 nm, less than 25 nm) and/or down to 20 nm (e.g., down to 15 nm, down to 10 nm, down to 5 nm, down to 4 nm, down to 3 nm, down to 2 nm, down to 1 nm, etc.) so that the species of interest 50 (e.g., a protein or peptide) is arranged linearly at the tip of the capillary. In one set of embodiments, the nanotip may have a cross-sectional dimension of between 1 nm and 5 nm.

Advantageously, such a linear arrangement may allow for exposure of individual bonds (e.g., peptide bonds) between the base fragments (e.g., amino acids) forming the species of interest, which may in turn facilitate fragmentation of the species of interest into its base fragments.

In some embodiments, the method comprises a step of fragmenting the species of interest (e.g., a protein) into base fragments (e.g., amino acids) by applying laser light to the species of interest within the capillary tip (e.g., nanotip). As shown in FIGS. 1A-1B, light 62 (e.g., a laser light), produced by the light source 15 (e.g., laser) adjacent the capillary tip 34 (e.g., the nanotip), may be applied to the solution 52 containing the species of interest 50. In some such embodiments, the species of interest is arranged linearly, such that the laser light is able to cleave the bonds between individual components (e.g., amino acids) from the species of interest (e.g., protein). For example, referring to FIG. 1B, the species of interest 50 may be arranged linearly within the capillary tip 34 (e.g., the nanotip), such that the light 62 (e.g., laser light) is able to cleave the bonds between individual components or molecules (e.g., amino acids) from the species of interest 50 (e.g., protein or peptide).

In some embodiments, upon fragmentation of the species of interest (e.g., protein) into its base fragments (e.g., amino acids), the base fragments may be emitted (e.g., ionized) from the opening at the nanotip. According to some embodiments, the nanotip may be sized such that the base fragments are emitted in a sequential fashion, e.g., preserving the order of the base fragments within the species of interest. For example, as shown in FIGS. 1A-1B, the based fragments 54 (e.g., amino acids) may be emitted from the opening 36 at the nanotip 34 (e.g., one by one) in a sequential order. In some embodiments, the emitted base fragments (e.g., amino acids) may be passed along to various components of the mass spectrometer (e.g., vacuum, ion optics, magnetic mass filter), and subsequently detected by one or more detectors. In some embodiments, the one or more detectors may be configured to determine a sequence of the species of interest (e.g., protein) by determining the emitted fragments (e.g., amino acids). Referring again to FIGS. 1A-1B as a non-limiting example, the emitted base fragments 54 (e.g., amino acids) may be passed along to various components of the mass spectrometer (e.g., vacuum 80, ion optics 100, mass filter 90 (e.g., magnetic mass filter)), and subsequently detected by one or more detectors 70. The one or more detectors 70 may be configured to determine a sequence of the species of interest (e.g., protein) by determining the emitted fragments 54 (e.g., amino acids). The specifics related to the various components and methods of emission, transmission, and detection of the base fragments are described in more detail below.

In some embodiments, the capillary may have a particularly advantageous configuration that allows for fragmentation of a species of interest within the capillary into individual components. A capillary having such a configuration may, for example, advantageously reduce intermixing and diffusion of the fragmented individual components within the capillary (e.g., within the nanotip of the capillary), and/or preserve the sequence of the individual components with respect to their original sequence in the species of interest prior to fragmentation, and/or allow for subsequent ionization of the fragmented individual components in a linear and sequential fashion at the tip of the capillary. For example, in one set of embodiments, the capillary may comprise a body portion and a tip portion (e.g., nanotip) adjacent the opening of the capillary fluidically connected to the body portion. For example, as shown in FIG. 1B, the capillary 30 may comprise a body portion 32 and a tip portion 34 (e.g., nanotip) adjacent the opening 36 of the capillary 30. The tip portion 34 may be fluidically connected to the body portion 32.

In some embodiments, the tip portion of the capillary may be substantially transparent to light having a certain wavelength or range of wavelengths emitted by the source of light. As used herein, a tip portion that is “substantially transparent” to a wavelength of light means that greater than 50% (e.g., greater than 60%, greater than 70%, greater than 80%, greater than 90%) and/or up to 95% (e.g., up to 99%, or up to 100%) of light having a certain wavelength or range of wavelengths can pass into the tip portion of the capillary. For example, the tip portion of the capillary (e.g., nanotip) may be transparent to light having a wavelength of less than or equal to 213 nm (e.g., less than or equal to 213 nm, less than or equal to 200 nm, less than or equal to 193 nm, less than or equal to 185 nm, less than or equal to 180 nm, less than or equal to 175 nm, less than or equal to 150 nm) and/or down to 170 nm (e.g., down to 160 nm, down to 155 nm, or down to 150 nm). The above-referenced values of wavelengths may have a deviation of +/−5 nm, of +/−10 nm, or +/−15 nm. Combination of the above-referenced ranges are also possible (e.g., less than or equal to 220 nm+/−5 nm and down to 150 nm+/−5 nm, less than or equal to 213 nm+/−5 nm and down to 150 nm+/−5 nm, less than or equal to 193 nm+/−5 nm and down to 160 nm+/−5 nm, or less than or equal to 185 nm+/−5 nm and down to 150 nm+/−5 nm). Other ranges are also possible.

In some embodiments, the body portion of the capillary may be substantially opaque to light having a certain wavelength or range of wavelengths emitted by the source of light, As used herein, a body portion that is “substantially opaque” to a wavelength of light means that less than 50% (e.g., less than 40%, less than 30%, less than 20%, less than 10%) and/or down to 5% (e.g., down to 1%, or down to 0%) of light having a certain wavelength or range of wavelengths can pass into the body portion of the capillary. For example, the body portion of the capillary may be opaque to light having a wavelength of less than or equal to equal to 213 nm (e.g., less than or equal to 200 nm, less than or equal to 193 nm, less than or equal to 185 nm, less than or equal to 180 nm, less than or equal to 175 nm, less than or equal to 150 nm) and/or down to 170 nm (e.g., down to 165 nm, down to 160 nm, down to 155 nm, or down to 150 nm). The above-referenced values of wavelengths may have a deviation of +/−5 nm, of +/−10 nm, or +/−15 nm. Combination of the above-referenced ranges are also possible (e.g., less than or equal to 220 nm+/−5 nm and down to 150 nm+/−5 nm, less than or equal to 213 nm+/−5 nm and down to 150 nm+/−5 nm, less than or equal to 193 nm+/−5 nm and down to 150 nm+/−5 nm, or less than or equal to 185 nm and down to 150 nm+/0 5 nm). Other ranges are also possible.

Referring to FIGS. 1A-1B as a non-limiting example, while the tip portion 34 (e.g., the nanotip) may be transparent to light having a wavelength in one or more ranges described above with respect to the tip portion of the capillary, the body portion 32 may be substantially opaque to light having a wavelength in one or more ranges described above with respect to the body portion of the capillary.

In some embodiments, while a majority of the species of interest contained within the tip portion (e.g., a substantially transparent tip portion) of the capillary is fragmented into individual components, a small amount, if any, of the species of interest contained within the body portion (e.g., a substantially opaque body portion) of the capillary is fragmented into individual components. For example, greater than 50% (e.g., greater than 60%, greater than 70%, greater than 80%, greater than 90%) and/or up to 95% (e.g., up to 99%, or up to 100%) of the species of interest contained within the tip portion of the capillary may be fragmented into individual components, e.g., due to the incident light. Additionally, less than 50% (e.g., less than 40%, less than 30%, less than 20%, less than 10%) and/or down to 5% (e.g., down to 1%, or down to 0%) of the species of interest contained within the body portion of the capillary may be fragmented into individual components.

For example, as shown in FIGS. 1A-1B, while a majority of the species of interest 50 (e.g., protein or peptide) contained within the transparent tip portion 34 of the capillary 30 is fragmented into individual components 54 (e.g., amino acids) by light 62, little, if any, of the species of interest 50 (e.g., protein or peptide) contained within the opaque body portion 32 of the capillary 30 is fragmented into individual components. In one set of embodiments, while all (e.g., 100%) of the species of interest contained within the tip portion of the capillary is fragmented by light from the source of light, little to no (e.g., 0%) species of interest contained within the body portion of the capillary is fragmented by light from the source of light.

In some embodiments, upon fragmentation of the species of interest into individual components at the tip portion of the capillary, the individual components may diffuse toward the opening of the tip portion and become ionized by an electrode proximate the capillary tip. The electrode may have any properties and/or configurations described elsewhere herein. For example, as shown in FIG. 1B, fragmented individual components 54 may diffuse toward the opening 36 of the tip portion 34 and become ionized by an electrode (not shown) proximate the capillary tip.

The capillary, in accordance with some embodiments, by having an opaque body portion and a transparent tip portion, may advantageously allow for fragmentation of the species of interest only at the transparent tip portion adjacent the opening of the capillary. As described elsewhere herein, the tip portion may be sized such that the species of interest contained within the tip portion is arranged in a substantially linear fashion. The capillary described herein may allow for fragmentation of the species of interest while arranged in a linear fashion in the tip portion, limit intermixing of the fragmented individual components prior to being ionized at the opening of the tip portion, and thus allow for ionization and detection of the fragmented individual components in their original sequence in the species of interest.

The tip portion of the capillary may have a size in one or more ranges described herein with respect to the opening of the capillary. In some embodiments, the tip portion comprises a cross-sectional dimension (e.g., a maximum cross-sectional dimension) of less than or equal to 150 nm, less than or equal to 130 nm, less than or equal to 125 nm, less than or equal to 120 nm, less than or equal to 110 nm, less than or equal to 100 nm, less than or equal to 90 nm, less than or equal to 80 nm, less than or equal to 75 nm, less than or equal to 70 nm, less than or equal to 65 nm, less than or equal to 60 nm, less than or equal to 55 nm, less than or equal to 50 nm, less than or equal to 45 nm, less than or equal to 40 nm, less than or equal to 35 nm, less than or equal to 30 nm, less than or equal to 25 nm, less than or equal to 20 nm, less than or equal to 15 nm, less than or equal to 10 nm, less than or equal to 5 nm, less than or equal to 3 nm, less than or equal to 2 nm, etc. In addition, the tip portion, in some cases, may have a cross-sectional dimension of at least 1 nm, at least 2 nm, at least 3 nm, at least 5 nm, at least 10 nm, at least 15 nm, at least 20 nm, at least 25 nm, at least 30 nm, at least 40 nm, at least 50 nm, at least 60 nm, at least 65 nm, at least 70 nm, at least 80 nm, at least 90 nm, etc. Combinations of these are also possible; for example, the opening may have a cross-sectional dimension of between 50 nm and 100 nm, or between 20 and 65 nm, between 1 nm and 5 nm, between 1 nm and 3 nm, etc. In some embodiments, the tip portion (e.g., nanotip) of the capillary may be nanosized, e.g., such as being formed from a nanotube. Non-limiting examples of nanotubes include a carbon nanotube and/or a nitride nanotube (e.g., a boron nitride nanotube). In some cases, the opening of the nanotip may have a cross-sectional dimension of between 1 nm and 5 nm (e.g., or between 1 nm and 3 nm).

In some embodiments, the body portion of the capillary may be fabricated from and/or coated with a material that is substantially opaque to a wavelength of light emitted by the light source. For example, the body portion of the capillary may be substantially opaque to light having a wavelength in one or more ranges described above with respect to the body portion of the capillary. In some cases, the capillary wall of the body portion may be constructed using an opaque material (e.g., a metal). Alternatively or additionally, the capillary wall may be formed from an originally transparent material that is subsequently coated with an opaque layer (e.g., a metal coating). Any of a variety of appropriate coating techniques may be employed to coat the body portion of the capillary.

In some embodiments, the capillary may comprise a capillary wall having a variable thickness along the length of the capillary. For example, the capillary wall may be the thinnest near the opening of the nanotip and may grow thicker further away from the nanotip. In one set of embodiments, the thickness of the capillary wall through which light passes through may grow substantially linearly as one moves further away from the nanotip (e.g., such as toward the capillary body). This may in turn lead to a decrease in the amount of light entering into the capillary as one moves away from the nanotip. In some embodiments, while the nanotip, which has a thinner capillary wall, is substantially transparent to a certain wavelength of light, the capillary body, which has a thicker capillary wall, may be substantially opaque to the wavelength of light.

In some cases, a particularly beneficial type of laser light and/or wavelength of light may be employed in the mass spectrometer. In some such embodiments, the light source is a UV light source and the laser light is ultraviolet light. In some cases, employing ultraviolet light, compared to other wavelengths of light (e.g., X-Ray), may lead to more favorable conditions for sequencing proteins. Such favorable conditions include, but are not limited to, more efficient fragmentation of a species of interest (e.g., a protein) into single molecules (e.g., single amino acids), the use of a lower power laser (thereby allowing more practical and safer operating), reduced heating of the fluid containing the species of interest (thereby reducing thermal degradation of the species), etc. In embodiments in which the species of interest is a protein, employing such a laser light and/or wavelength of laser light may result in a relatively high probability of scission at the peptide backbones, thereby allowing formation of individual amino acids. In some cases, other types of light sources (e.g., IR) having a different wavelength may also be employed.

In some embodiments, the light source is configured to a produce light at any of a variety of appropriate wavelengths. In some embodiments, the light source may have a wavelength of at least 150 nm (e.g., at least 155 nm, at least 157 nm, at least 160 nm, at least 165 nm, at least 170 nm, at least 175 nm, at least 180 nm, at least 185 nm, at least 190 nm, at least 193 nm, at least 195 nm, at least 200 nm, at least 210 nm, at least 213 nm, at least 220 nm, etc.). In some embodiments, the light source may have a wavelength of no more than 230 nm (e.g., no more than or equal to 222 nm, no more than 220 nm, no more than 213 nm, no more than 210 nm, no more than 200 nm, no more than 195 nm, no more than 193 nm, no more than 190 nm, no more than 185 nm, no more than 180 nm, no more than 175 nm, no more than 165 nm, no more than 160 nm, no more than 157 nm. The above-referenced values of wavelengths may have a deviation of +/−5 nm, of +/−10 nm, or +/−15 nm. Any of the above-referenced ranges may be possible (e.g., at least 150 nm+/−5 nm and no more than 213 nm+/−5 nm, at least 160 nm+/−5 nm and no more than 213 nm+/−5 nm, or at least 157 nm+/−5 nm and no more than 193 nm+/−5 nm, at least 180 nm+/−5 nm and no more than 213 nm+/−5 nm, at least 193 nm+/−5 nm and no more than 213 nm+/−5 nm). Other ranges are also possible.

In some embodiments, by employing a light source described herein, a relatively high fragmentation efficiency may be achieved. The term fragmentation efficiency, as used herein, refers to the probability that a certain base fragment (e.g., a specific amino acid) can be cleaved from the species of interest as a single molecule. In some such embodiments, for the majority of amino acids, the fragmentation efficiency is between 60% to 95% or between 65% to 92%.

In addition, the present disclosure generally relates in certain embodiments to the creation of ionized molecules, e.g., for detection in a mass spectrometer, or for other uses such as lithography, sputtering machines, propulsion, etc. Some embodiments include an ion source comprising a capillary tip that may allow for direct ion evaporation of samples with an applied electric field. In some cases, the tip may have an opening with a cross-sectional dimension (e.g., diameter) less than 125 nm or 100 nm, etc. In addition, certain aspects are directed to using a capillary tip that allow for detection of samples (e.g., amino acids), and in some cases allows for sequencing. For instance, some embodiments are directed to allowing single ions and ionic clusters to be evaporated at a high rate directly from aqueous samples in a mass spectrometer. Other aspects are directed to methods for making or using such ionized molecules, methods for making or using devices to create such ionized molecules, or the like.

For example, some embodiments are generally directed to an ion source comprising a capillary and an electrode, which may be annular in some cases, between which a voltage is applied to produce ions. In some cases, the capillary may have an inner tip diameter of less than 125 nm or 100 nm, etc. This may allow ions to evaporate directly off of the meniscus of a fluid in the capillary, bypassing the wasteful droplet evaporation process. In this regime, ion evaporation may account for the majority of the ionic current, and this emission mode can be achieved in some cases with relatively low salinity solutions. In some embodiments, tips with inner diameters less than 125 nm or 100 nm (e.g., less than or equal to 65 nm or 60 nm, etc.) may be able to produce a high fraction of bare ions or ionic clusters, for example, comprising small numbers of solvent molecules, e.g., only 1 or 2 solvent molecules. The small area of the liquid vacuum interface may in some cases prevent significant evaporative heat loss, which allows the use of volatile solvents like water in certain cases. Methods such as these could be used in some embodiments to analyze molecules or ions, e.g., biomolecules such as amino acids, nucleic acids, peptides or proteins, etc. In some cases, ion sources such as those described herein may improve the sensitivity of mass spectrometry experiments, allow single-molecule protein sequencing or single cell proteomic studies. Other applications such as those described below are also possible.

For example, some embodiments are generally directed to an ion source comprising a capillary and an electrode. The electrode may be used to generate ionized molecules directly from a fluid within the capillary, e.g., into a reduced pressure environment or vacuum, e.g., at a pressure of 100 mPa, or other pressures described herein. In certain embodiments, the opening of the capillary is sized such that, when an electric field is applied, a fluid within the capillary forms a charged meniscus and species within the fluid exit the charged meniscus, e.g., via predominately ion evaporation. The use of capillaries with a submicron opening (e.g., less than 125 nm or 100 nm, etc.) may favor the ionization of a fluid via ion evaporation, where the species exiting the capillary directly ionizes into single charged ions or charged ion clusters, in contrast to electrospray ionization, where the species exiting the capillary exit via a liquid jet that breaks up into charged droplets that further break down into charged ions in the presence of a background gas, although it should be understood that some electrospray ionization may still occur in some cases. Ion evaporation may be preferred in certain applications, e.g., that require the efficient use or generation of single ions from a fluid. For example, certain embodiments are related to ion sources in mass spectroscopy, where single charged ions can be directly generated and subsequently detected.

According to one set of embodiments, the ion source comprises a capillary defining an opening having a cross-sectional dimension (e.g., inner diameter of the capillary) of less than 100 nm. The opening may also be sized in some cases such that when an electric field is applied, ion evaporation dominates over liquid jet formation. For instance, in certain embodiments, at least 50% of the exiting species may exit via ion evaporation or in the form of ions or ion clusters. For instance, a nanoscale capillary can allow ions to evaporate directly off of a fluid meniscus. In some embodiments, a fluid can be passed into a capillary having such an opening, and directly delivered into a reduced pressure or vacuum environment (e.g., having a pressure of no more than 100 mPa) in the form of ions and ion clusters. The ions and ion clusters can be analyzed by a mass filter and an ion detector in a mass spectrometer, or applied to other applications such as those described herein.

In addition, certain aspects are related to methods of sequencing molecules or polymers, such as biopolymers, from a fluid within the capillary (e.g., via mass spectrometry). In some embodiments, the fluid comprises a polymer, such as a biopolymer, dissolved in a solvent having relatively high vapor pressure (e.g., water). Typically, the evaporation of a high volatility solvent (e.g., water) can lead to freezing of the fluid at the opening of the capillary as the solvent evaporates, thus limiting the mass spectrometer's ability to successfully evaporate the ions from the fluid. However, the opening of the capillary may be sized such that the fluid meniscus at the opening, e.g., as discussed herein, may have a smaller area that may reduce these effects. Thus, the use of capillary with small openings in the ion source of a mass spectrometer can allow the study of molecules, for example, polymers or biopolymers such as amino acids, nucleic acids, and peptides or proteins, in aqueous solutions. Molecules that are not polymers may also be studied in certain embodiments.

To sequence a molecule such as a polymer (e.g., a biopolymer), certain embodiments are directed to applying an electric field to ionize the molecule proximate the opening of a capillary to produce ionized fragments. In certain embodiments, the ionized fragments from the fluid are directly passed into a reduced pressure environment. The ionized fragments in some cases may comprise single ions or ion clusters, as discussed herein, for instance, having a small number of solvent molecules (e.g., water). The opening may be sized such that ionized fragments exit the opening in a sequential order according to the sequence of the molecule. For instance, certain embodiments allow for the determination of a sequence of the molecule by determining ionized fragments produced by ionizing the molecule within the detector.

In addition, certain aspects are directed to devices comprising an ion source having a capillary as disclosed herein. The device may also have an electrode proximate the capillary. It should be noted that although some embodiments disclose the use of the ion source in a mass spectrometer, the use of the ion source as disclosed herein does not apply solely to a mass spectrometer. The ion source could also be used, for example, in lithography machines, sputtering machines, space propulsion systems, etc., as discussed herein.

Certain aspects are directed to an ion source comprising a capillary defining an opening and an electrode posited proximate the opening. The capillary may have an opening at an end or a tip of the capillary. The opening may have any of a variety of cross-sectional dimensions, and may be of any shape, e.g., circular, elliptical, square, etc. In some embodiments, the opening comprises a cross-sectional dimension of less than or equal to 150 nm, less than or equal to 130 nm, less than or equal to 125 nm, less than or equal to 120 nm, less than or equal to 110 nm, less than or equal to 100 nm, less than or equal to 90 nm, less than or equal to 80 nm, less than or equal to 75 nm, less than or equal to 70 nm, less than or equal to 65 nm, less than or equal to 60 nm, less than or equal to 55 nm, less than or equal to 50 nm, less than or equal to 45 nm, less than or equal to 40 nm, less than or equal to 35 nm, less than or equal to 30 nm, less than or equal to 25 nm, less than or equal to 20 nm, less than or equal to 15 nm, less than or equal to 10 nm, less than or equal to 5 nm, less than or equal to 2 nm, etc. In addition, the opening, in some cases, may have a cross-sectional dimension of at least 1 nm, at least 5 nm, at least 10 nm, at least 15 nm, at least 20 nm, at least 25 nm, at least 30 nm, at least 40 nm, at least 50 nm, at least 60 nm, at least 65 nm, at least 70 nm, at least 80 nm, at least 90 nm, etc. Combinations of these are also possible; for example, the opening may have a cross-sectional dimension of between 50 nm and 100 nm, between 20 and 65 nm, between 1 nm and 5 nm, or between 1 nm and 3 nm, etc. While the above embodiment describes a capillary having an opening at the end or the tip of the capillary, it should be understood that not all embodiments described herein are so limiting, and in certain embodiments, the capillary may additionally or alternatively have a plurality of openings along the side of the capillary. In addition, in some cases, a device may have one or more apertures or openings, e.g., in a channel or other structure. Thus, an opening need not be the opening of a capillary.

In some embodiments, the capillary is tapered at the opening. For instance, the capillary may have a constant tapering, e.g., such that the tip of the capillary is cone-shaped. Any suitable angle may be present. For example, the angle may be less than 15 degrees, less than 10 degrees, less than 9 degrees, less than 8 degrees, less than 7 degrees, less than 6 degrees, less than 5 degrees, less than 4 degrees, less than 3 degrees, less than 2 degrees, or less than 1 degree (where 0 degrees indicates no taper, i.e., the capillary is cylindrical. In addition, in some cases, the angle of the taper may be at least 1 degree, at least 3 degrees, at least 5 degrees, etc., in certain cases. Combinations of these ranges are also possible, e.g., the tapering may be between 1 degree and 5 degrees.

In certain embodiments where the capillary is tapered at the opening, a laser pulling technique can be used to fabricate the tapered opening. It should be understood that techniques other than a laser-pulling technique could also be used to produce capillaries with tapered openings. It should also be understood that, although the capillary discusses herein has a tapered opening, in other examples, the opening of the capillary could be non-tapered.

The capillary of the ion source comprises quartz in certain embodiments. Additional examples of materials that can be used to fabricate the capillary include, but are not limited to, glass (e.g., borosilicate glass), a plastic, a metal, a ceramic, a semiconductor, a carbon nanotube, a boron nitride nanotube, etc.

In some embodiments, the capillary has a relatively high aspect ratio, e.g., a ratio of the length of the capillary to the cross-sectional dimension (e.g., diameter) of the capillary's opening. For example, the capillary may have an aspect ratio that is greater than 10,000. However, it should be understood that the aspect ratio is not so limited. For instance, in some examples, the aspect ratio of the capillary length to the opening's cross-sectional dimension may be greater than 10, greater than 100, greater than 1,000, greater than 10,000, greater than 100,000, or greater than 1,000,000.

The capillary may have a circular or a non-circular cross-section (e.g., square). In addition, in some embodiments, the capillary may have a relatively small cross-section, e.g., diameter. For instance, the cross-sectional dimension of the capillary may be less 200 nm, less than 150 nm, less than 75 nm, less than 60 nm, less than 50 nm.

Certain embodiments of the ion source also comprise an electrode positioned proximate the capillary, e.g., the opening of the capillary. The electrode may be used to apply an electric field (for example, as described below) to a fluid within the capillary, e.g., to be applied to the meniscus. In some cases, the fluid within the capillary may be in contact with a counterelectrode, e.g., such that a voltage difference between the electrode proximate the opening of the capillary and the counterelectrode within the capillary is able to generate an electric field to the fluid. In some embodiments, the electrode may be positioned so as to cause an electric field maximum proximate the opening of the capillary. For example, in some embodiments, the electrode may be positioned within 50 mm, within 40 mm, within 30 mm, within 20 mm, within 15 mm, within 10 mm, within 5 mm, within 3 mm, within 2 mm, within 1 mm, etc., of the opening of the capillary.

The electrode, in some embodiments, may be positioned around the capillary or the nanotip, or may be positioned in front of the capillary or the nanotip, e.g., in front of the opening of the capillary, or in a downstream direction. For example, referring to FIGS. 1A-1B as a non-limiting example, an electrode (not shown) may be positioned around the capillary 30 or the nanotip 34, in front of the capillary 30 or the nanotip 34, in front of the opening 36 of the capillary 30, or in a direction downstream the capillary 30.

The electrode may have any suitable shape. In some cases, the electrode is circular or circularly symmetric, or is symmetrically positioned with respect to the capillary. However, other shapes or arrangements are also possible.

In some embodiments, the electrode defines an opening (e.g., an aperture). Thus, the electrode may be annular in some cases. The electrode may be positioned such that ions or ion clusters escaping the fluid in the capillary pass through the center opening of the electrode. The center opening of the electrode can be of any shape, including, but not limited to, a circular shape that can be positioned annularly around the opening of the capillary. The opening may also be non-circular in some cases. In some embodiments, the opening of the electrode is positioned coaxially to the opening of the capillary. That is, the opening can be aligned, in certain embodiments, to the opening of the capillary, e.g., such that an imaginary line passing through the center of a cross-section of the capillary passes through the center opening of the electrode. This may facilitate the application of an electric field to the fluid in the capillary, e.g., to cause ions or ion clusters to exit the fluid, as discussed herein.

For example, in some embodiments, the electrode has a center opening with cross-sectional dimension (e.g., inner diameter) greater than the cross-sectional dimension of the opening of the capillary, e.g., at the end or tip of the capillary. For instance, in accordance to certain embodiments, the electrode has a center opening with a cross-sectional dimension (e.g., inner diameter) at least 5 times greater than the cross-sectional dimension of a capillary's opening. However, it should be understood that the ratio of the cross-sectional dimensions of the electrode's center opening to the capillary's opening is not limited. For instance, in some examples, the cross-sectional dimension of the center opening of the electrode could be at least 2 times, at least 3 times, at least 5 times, at least 10 times, at least 20 times, at least 30 times, at least 50 times, at least 75 times or at least 100 times larger than the cross-sectional dimension of the capillary's opening. In certain cases, the opening of the electrode may have a cross-sectional dimension of less than 10 cm, less than 5 cm, less than 3 cm, less than 1 cm, less than 5 mm, less than 3 mm, less than 1 mm, etc. In addition, in some embodiments, a front side of the electrode is positioned in front of the opening of the capillary.

In addition, the electrode itself can be of any shape (e.g., circular or non-circular). The electrode may have the same or a different shape than its opening (if present). The electrode may have any suitable cross-sectional dimension. For example, the electrode may have a cross-sectional dimension of less than 10 cm, less than 5 cm, less than 3 cm, less than 1 cm, less than 5 mm, less than 3 mm, less than 1 mm, etc.

In some embodiments, the electrode comprises steel. Other examples include copper, graphite, silver, aluminum, gold, electrically conducting ceramics, or the like.

Thus, certain embodiments are directed to an electrode able to generate an electric field. In some cases, as noted, the electrode may be positioned to create an electric field maximum proximate the opening of the capillary. In some embodiments, a fluid is housed in the capillary such that when an electric field is applied by the electrode proximate the opening of the capillary, molecules within the fluid can ionize and exit from the opening of the capillary, e.g., as ions or ion clusters such as discussed herein. In some cases, for example, the electrode and the capillary (e.g., the interior of the capillary) may be connectable to a voltage source, e.g., as discussed herein.

Thus, in certain embodiments, the voltage source, in conjunction with the electrodes, may be used to produce an electric field to cause ions or ion clusters to exit a fluid in the capillary, e.g., as discussed herein. In some embodiments, a voltage is applied to generate an electric field at least sufficient to ionize molecules within the fluid at the opening of the capillary, e.g., to produce ions or ion clusters. For instances, in certain embodiments, a voltage in the range of 80 V to 400 V could be used to generate an electric field. In some cases, the voltage may be at least 40 V, at least 60 V, at least 80 V, at least 100 V, at least 120 V, at least 140 V, at least 160 V, at least 180 V, at least 200 V, at least 220 V, at least 240 V, at least 260 V, at least 280 V, at least 300 V, at least 320 V, at least 340 V, at least 360 V, at least 380 V, at least 400 V, at least 450 V, at least 500 V, at least 600 V, etc. In addition, in some cases, the voltage may be no more than 600 V, no more than 500 V, no more than 450 V, no more than 400 V, no more than 380 V, no more than 360 V, no more than 340 V, no more than 320 V, no more than 300 V, no more than 280 V, no more than 260 V, no more than 240 V, no more than 220 V, no more than 200 V, no more than 180 V, no more than 160 V, no more than 140 V, no more than 120 V, no more than 100 V, no more than 80 V, no more than 60 V, etc. In some cases, combinations of these voltages are possible. For instance, the voltage may be applied between 80 V and 360 V, etc. The voltage may be applied as a constant voltage, or a varying or periodic voltage in certain cases.

As mentioned, a voltage may be applied to create an electric field maximum proximate the opening of the capillary, or the fluid within the capillary (e.g., at the meniscus at the opening). For example, a voltage may be applied to create an electric field maximum of at least 0.5 V/nm, at least 0.7 V/nm, at least 1 V/nm, at least 1.1 V/nm, at least 1.3 V/nm, at least 1.5 V/nm, at least 2 V/nm, at least 2.5 V/nm, at least 3 V/nm, at least 3.5 V/nm, at least 4 V/nm, etc. In certain embodiments, the electric field maximum may be no more than 5 V/nm, no more than 4.5 V/nm, no more than 4 V/nm, no more than 3.5 V/nm, no more than 3 V/nm, no more than 2.5 V/nm, no more than 2 V/nm, no more than 1.5 V/nm, no more than 1 V/nm. Combinations of these ranges are also possible in some embodiments; for example, the electric field may be between 1.5 V/nm and 3.0 V/nm, between 1.5 V/nm and 4.0 V/nm, etc.

Without wishing to be bound by any theory, it is believed that in certain embodiments, when the electric field is applied, fluid within the capillary forms a charged meniscus and species exit the charged meniscus, e.g., as ions or ion clusters. In some cases, the opening of the capillary may be sized such that at least 10% of the exiting species exit via ion evaporation, e.g., as ions or ion clusters. In some cases, at least 20%, at least 30%, at least 40%, at least 50%, at least 60%, at least 70%, at least 80%, at least 90%, at least 95%, etc. of the exiting species exit via ion evaporation.

As described previously, according to certain embodiments, a charged fluid meniscus in the shape of a cone can be generated at the opening of a capillary under an electric field. In some embodiments, the conical fluid meniscus acts as a point source to allow species to exit as ions or ion clusters.

The fluid meniscus could produce exiting species via mechanisms such as charged droplets via electrospray ionization, and/or ions and ion clusters via ion evaporation. However, in the case of electrospray ionization, the exiting species exiting from the liquid meniscus would exit as charged droplets of fluid containing the exiting species, which would require the presence of a background gas to further break down the droplets into individual ions, typically via a Coulomb fission process. Ion evaporation, on the other hand, describes a process where a molecule is directly ionized into ions (e.g., bare ions) or ion clusters (e.g., ions with solvent molecules), instead of charged droplets. An ion cluster may contain a single ion and a plurality of solvent molecules, usually a relatively small number. For example, the ion clusters may contain no more than 10, no more than 9, no more than 8, no more than 7, no more than 6, no more than 5, no more than 4, no more than 3, no more than 2, or no more than 1 solvent molecule.

Thus, for example, in some embodiments, the opening of the capillary is sized (e.g., the cross-sectional dimension of the opening is less than 125 nm or 100 nm, etc.) such that the formation of charged droplets can be avoided, and such that at least 50% (e.g., at least 60%, at least 70%, at least 80%, at least 95%, at least 90%, at least 93%, at least 99%, or all) of the exiting species directly ionize as ions or ion clusters from conical fluid meniscus at the opening of the capillary.

As mentioned, in some embodiments, a capillary having a relatively small opening (e.g., a cross-sectional dimension of less than 125 nm or 100 nm, etc.) may be associated with the production of a relatively small number of solvent molecules in an ion cluster, e.g., as described above. In some embodiments, the opening of the capillary may be sized (e.g., less than 125 nm or 100 nm, etc.) such that the plurality of solvent molecules comprises less than or equal a certain number of solvent molecules, e.g., such that on average, the ion clusters produced by the ion source contain less than or equal to 7, 6, 5, 4, 3, or 2 solvent molecules. In some embodiments, a substantial number (e.g., greater than or equal to 50%, greater than or equal to 60%, greater than or equal to 70%, greater than or equal to 80%, greater than or equal to 85%, greater than or equal to 90%, greater than or equal to 95%, greater than or equal to 99%, or all) of the ion clusters contains one or two solvent molecules.

In some embodiments, a voltage may be applied to the tip of the capillary to generate a current. In some embodiments, the tip of the capillary may have a relatively low current. In some embodiments, the current at the tip of the capillary may be at least 0.1 pA (e.g., at least 0.5 pA, at least 1 pA, at least 2 pA, at least 3 pA, at least 5 pA, at least 10 pA, at least 15 pA, at least 20 pA, at least 50 pA, at least 100 pA, at least 150 pA, at least 200 pA, at 500 pA, at least 1 nA, etc.). In some embodiments, the tips of the capillary may be no more than 2 nA (e.g., no more than 1 nA, no more than 500 pA, no more than 200 pA, no more than 150 pA, no more than 100 pA, no more than 10 pA, no more than 20 pA, no more than 18 pA, no more than 15 pA, no more than 10 pA, no more than 5 pA, no more than 3 pA, no more than 2 pA, no more than 1 pA, no more than 0.5 pA, etc.). Any of the above-referenced ranges are possible (e.g., at least 0.1 pA and no more than 2 nA, or at least 3 pA and no more than 20 pA). Other ranges are also possible.

In addition, as discussed, certain aspects are directed to methods of ionizing a fluid using an ion source, e.g., to produce single ions or ion clusters. Certain embodiments comprise passing a fluid into a capillary defining an opening having a cross-sectional dimension less than 125 or 100 nm, etc., or other configurations such as those discussed herein.

In some embodiments, the fluid comprises a sample and a solvent. The sample may include any species of interest that can be ionized from the opening of the capillary. For instance, in accordance with certain embodiments, a species of interest comprises a biopolymer (e.g., nucleic acids such as DNA or RNA, peptides or proteins, etc.). Other examples include other types of polymers, e.g., nylon, polyethylene, etc., or other species of interest that are not necessarily polymers, e.g., biomolecules. Non-limiting examples of biomolecules may include monomers such as amino acids, nucleotides, etc. In some cases, the species of interest is unknown, and it is desired that the structure of the species be at least partially determined, e.g., by ionizing the species and detecting the ion fragments, such as in mass spectroscopy or other related techniques.

In some embodiments, the solvent may be any liquid that can be used to dissolve or suspend the sample or the species of interest. For instance, in accordance with certain embodiments, the solvent comprises water. However, the solvent is not limited to water. In some cases, the solvent may be an aqueous solution, e.g., having any of a variety of salt concentrations. In some embodiments, an aqueous solution may have a salt concentration of greater than or equal to 10 mM, greater than or equal to 20 mM, greater than or equal to 30 mM, greater than or equal to 50 mM, greater than or equal to 100 mM, greater than or equal to 150 mM, greater than or equal to 200 mM, greater than or equal to 300 mM, greater than or equal to 400 mM, greater than or equal to 500 mM, greater than or equal to 750 mM, greater than or equal to 1 M, greater than or equal to 2 M, greater than or equal to 5 M, or greater than or equal to 7.5 M. In some embodiments, an aqueous solution may have a salt concentration of less than or equal to 10 M, less than or equal to 7.5 M, less than or equal to 5 M, less than or equal to 2 M, less than or equal to 1 M, less than or equal to 750 mM, less than or equal to 500 mM, less than or equal to 400 mM, less than or equal to 200 mM, less than or equal to 150 mM, less than or equal to 100 mM, less than or equal to 50 mM, less than or equal to 30 mM, less than or equal to 20 mM, less than or equal to 10 mM, etc. Combination of the above-referenced ranges are possible (e.g., greater than or equal to 100 mM and less than or equal to 10 M, or greater than or equal to 150 mM and less than or equal to 1 M).

Additional examples of solvents that can be used include, but are not limited to, formamide, alcohols (e.g., ethanol, isopropanol, etc.), organic solvents (e.g., toluene, acetonitrile, acetone, hexane, etc.), ionic liquids, inorganic solvents (e.g., ammonia, sulfuryl chloride fluoride, liquid acids and bases, etc.). Combinations of any of these and/or other solvents are also possible in certain cases.

In some embodiments, the fluid comprises a solvent having a relatively low pH value. For example, in some embodiments, the solvent may have a pH of at least 3 (e.g., at least 3.1, at least 3.2, at least 3.4, at least 3.6, at least 3.8, etc.). Additionally, in some embodiments, the solvent may have a pH of no more than 4 (e.g., no more than 3.9, no more than 3.8, no more than 3.6, no more than 3.4, no more than 3.2, no more than 3.1, etc.). Combination of the above-referenced ranges are possible (e.g., at least 3 and no more than 4). Other ranges are possible.

In addition, in some embodiments, the fluid comprises a solvent (e.g., water) having a relatively high volatility, e.g., to facilitate the production of ions or ion clusters. For instance, water, with its boiling point of 100° C., can be considered to be volatile in some cases. In some embodiments, liquids with boiling points close to room temperature could be used to facilitate the production of ions or ion clusters. In some embodiments, a solvent that could be used to facilitate the production of ions or ion clusters may have a boiling point of less than or equal to 100° C., less than or equal to 80° C., less than or equal to 60° C., less than or equal to 40° C., less than or equal 20° C., etc. In addition, the solvent may have a boiling point greater than or equal to 10° C., greater than or equal to 30° C., greater than or equal to 50° C., greater than or equal to 70° C., greater than or equal to 90° C., etc. Combination of these are also possible; for example, the solvent may have a boiling point of between 50° C. and 100° C. Additional examples of solvents having a relatively high volatility include, but are not limited to, acetone, isopropanol, hexane, etc.

In some embodiments, the temperature of the capillary (in addition to the type of fluid it contains) may be varied to control the number of solvent molecules in a resultant ion cluster. In some embodiments, the temperature of the capillary is set such that the plurality of solvent molecules comprises less than or equal a certain number of solvent molecules, e.g., such that on average, the ion clusters produced by the ion source contain less than or equal to 7, 6, 5, 4, 3, or 2 solvent molecules. In some embodiments, the temperature is at least 20° C., at least 30° C., at least 40° C., at least 50° C., at least 60° C., or at least 70° C. In some embodiments, the temperature is no more than 80° C., no more than 70° C., no more than 60° C., no more than 50° C., no more than 40° C., no more than 30° C. Combination of the above-referenced ranges are possible (e.g., greater than or equal to 20° C. and less than or equal to 80° C.). In some cases, the temperature of the capillary is controlled by a resistive heater, by a Peltier junction, by an infrared heater, etc.

In some embodiments, an appropriate range of electric field and an appropriate range of sizes of the capillary opening can be selected to cause at least some of the molecules to exit as ions or ion clusters, e.g., as discussed herein.

Certain embodiments comprise passing the ionized molecules from the fluid directly into a reduced pressure or vacuum environment. Without wishing to be bound by any theory, it is noted that techniques such as electrospray ionization typically requires the presence of a background gas to further break down the droplets into individual ions, typically via a Coulomb fission process. In contrast, in accordance with certain embodiments, ions or ion clusters produced as discussed herein can be directly passed into such an environment, without requiring substantial amounts of background gas. Thus, certain techniques such as mass spectrometry may be performed using a reduced pressure or vacuum environment, without necessarily requiring the addition of a background gas.

Thus, in one set of embodiments, the capillary may be positioned to allow ions or ion clusters exiting the opening to enter a reduce pressure or vacuum environment. In some cases, the environment may be an environment having a pressure of no more than 100 mPa. In certain embodiments, the environment may have a pressure of no more than 1000 mPa, no more than 300 mPa, no more than 100 mPa, no more than 30 mPa, no more than 10 mPa, no more than 3 mPa, no more than 1.5 mPa, no more than 1 mPa, no more than 0.3 mPa, no more than 0.1 mPa, etc. In some embodiments, the ions or ion clusters from the fluid are passed directly into a vacuum environment.

It should be understood that some of the embodiments provided herein focused on passing the ionized molecules from the fluid directly into an environment having a pressure of no more than 100 mPa. However, it should be understood the pressure within the environment is not limited to 100 mPa. In some embodiments, the pressure could also be greater than or equal to 100 mPa, and less than or equal to 1 Pa.

In some embodiments, the mass spectrometer comprises a pump. The pump may be used to create a reduced pressure or vacuum environment, e.g., as discussed herein. Non-limiting examples of pumps include diffusion pumps, molecular drag pumps, turbomolecular pumps, or the like.

In some embodiments, there may be a relatively high pressure difference between the vacuum chamber and the fluid at the capillary opening. For instance, the pressure may be about 1 atmosphere at where the fluid enters in the capillary and about 100 mPa, or other reduced pressures such as those described herein, inside the vacuum chamber where the opening of the capillary is located. However, in some cases such as are described herein, the fluid meniscus at the opening of the capillary may be relatively stable despite the relatively high pressure difference, e.g., due to the surface tension of the fluid at the meniscus. For instance, the pressure difference across the fluid meniscus at the opening of the capillary may be at least 0.1 atm, at least 0.2 atm, at least 0.3 atm, at least 0.4 atm, at least 0.5 atm, at least 0.6 atm, at least 0.7 atm, at least 0.8 atm, at least 0.9 atm, at least 1 atm, etc. Furthermore, in some embodiments, the hydraulic resistance of a fluid in a capillary such as described herein (e.g., a capillary with an opening less than 100 nm) may be higher than that in an ion source employed in electrospray ionization.

In accordance with certain embodiments, the opening of the capillary is sized such that a solvent having a relatively high volatility remain unfrozen at the opening of the capillary when exposed to relatively low pressures. In some embodiments, the opening of the capillary is small enough such that a solvent of relatively high volatility remains unfrozen as the solvent enters the surrounding environment. In some embodiments, the opening of the capillary is small enough such that a fluid comprising a sample and a solvent remains unfrozen as the species of interest ionizes, such that at least some of the species of interest ionizes to form ions (e.g., single ions) or ion clusters.

Some aspects are directed to a mass spectrometer comprising an ion source as described herein. However, ion sources such as described herein are not limited to only mass spectrometers, but can be used in other applications, such as lithography, sputtering machines, propulsion (e.g., space propulsion), etc. As a non-limiting example, in lithography, focused ion beam (FIB) machines can be used to examine and/or modify lithography masks, and/or to etch features into materials by sputtering. Sputtering is a process by which atoms are removed from the surface of a solid by ions that impinge with high kinetic energy. In some embodiments, the ion source described herein is present in a focused ion beam (FIB) machine, which may be used to deliver molecules to a substrate material in a patterned fashion.

In certain embodiments, an ion source as described herein may be used with a liquid-chromatography mass spectrometry system. For instance, a liquid chromatograph can be coupled with an ion source to separate peptides or other molecules before ionizing and delivering them into a mass spectrometer. In some cases, the mass spectrometer may be used to perform a single or tandem (MS/MS) analysis to identify the ionized peptides or molecules, as in a proteomics experiment. Advantageously, the use of the ion source (having a capillary with a nanosized opening and/or tip) described herein to deliver ions directly into a low pressure environment may improve the sensitivity of the instrument, the ion transmission efficiency in such a system, and remove the need for multiple pumping stages.

In certain embodiments, an ion source as described herein may be used as both a nanopipette and an ion source. For instance, a capillary (e.g., a pulled quartz capillary) described herein having a nanosized tip may be used to puncture a cell or tissue and withdraw its biomolecular contents. The capillary may then be directly inserted into a vacuum chamber (e.g., having relatively high vacuums, such as those having reduced pressures such as those described herein) and the extracted molecules may be ionized and delivered to a mass spectrometer. Such techniques may be used, for example, to sample relatively small liquid volumes, such as the contents of a single cell. For instance, such a technique may be used for single cell proteomics studies.

As another example, in some embodiments, the ion source described herein is used for propulsion. For instance, forward propulsion of an object may be generated when ions are ejected in the backward direction. In some embodiments, an ion source such as described herein is used in a propulsion system. This can be used, for example, to deliver a high thrust relative to the weight of the ion source due to, e.g., the small size of the ion source. Additionally, in some cases, the propulsion systems can be made compact and consume relatively less fuel compared to conventional propulsion systems.

In addition, some aspects are directed to a mass spectrometer comprising an ion source as described herein. In some cases, the mass spectrometer may include, besides an ion source such as described herein, components such as vacuum chambers (e.g., able to produce any of the reduced pressures described herein), ion optics (e.g., one or more lenses such as Einzel lenses, etc.), mass filters (e.g., quadrupole mass filters, magnetic sector mass filters, etc.), detectors, ion benders, ion traps, or the like. Examples of specific detectors include, but are not limited to, Faraday cups, electron multipliers, dynodes, charge coupled devices (CCDs), CMOS sensors, and phosphor screens, etc. Additional non-limiting examples of mass spectrometers are described in a provisional application filed on Apr. 23, 2021, entitled “Systems and Methods for Single-Ion Mass Spectrometry with Temporal Information,” incorporated herein by reference in its entirety.

In addition to an ion source, various ion optics can be positioned downstream of the ion source such that the exiting molecules (e.g., ions and ion clusters) can be transported along a path downstream of the ion source in certain cases, e.g., the downstream direction is the direction in which ions or ion clusters travel. Referring again to FIGS. 1A-1B as a non-limiting example, an ion optics 100 may be positioned downstream the ion source 20, such that the exiting molecules 54 may be transported along a path downstream of the ion source 20. Those of ordinary skill in the art will be familiar with various ion optics used in mass spectrometry. In some embodiments, the ion optics comprises one or more Einzel lenses (e.g., a first Einzel lens and a second Einzel lens). As the ion optics transmit the molecules (e.g., ions or ion clusters) to the mass filter, the mass-to-charge ratios (m/z) of molecules (e.g., ions and ion clusters) may be analyzed by the mass filter. In some cases, the mass filter may be positioned downstream the ion optics. For example, as shown in FIGS. 1A-1B, the mass filter 100 (e.g., magnetic mass filter) may be positioned downstream the ion optics 100. Examples of mass filters include, but are not limited to, quadrupole mass filters, magnetic sector mass filters, etc.

In some embodiments, one or more detector(s) may be positioned further downstream of the mass filter. Referring again to FIGS. 1A-1B as a non-limiting example, one or more detectors 70 may be positioned downstream the magnetic filter 90. The detector may be any suitable detector able to detect ions or ion clusters. In some embodiments, ions and ion clusters with mass-to-charge ratios (m/z) within an acceptance window of the mass filter are passed to an ion bender. The ion bender may be configured to deflect the ions and ion clusters leaving the mass filter to a detector. For instance, as a non-limiting example, ions or ion clusters are passed from an ion bender to a detector. In some embodiments, the detector can be used to determine the ions or ion clusters.

In some embodiments, a mass spectrometer such as described herein may comprise a mean or overall ion transmission (e.g., ratio of ions and ion clusters detected to the ions and ion clusters exiting from the fluid at the opening of the capillary) of greater than 0.01, and in some cases, at transmissions of at least 0.02, at least 0.03, at least 0.05, at least 0.1, at least 0.15, at least 0.2, at least 0.3, at least 0.4, at least 0.5, at least 0.6, at least 0.7, at least 0.75, at least 0.8, at least 0.9, at least 0.93, at least 0.95, at least 0.99, etc. In some cases, the overall ion transmission is no more than 1, no more than 0.99, no more than 0.95, no more than 0.93, no more than 0.9, no more than 0.8, no more than 0.75, no more than 0.7, no more than 0.6, no more than 0.5, no more than 0.4, no more than 0.3, no more than 0.2, no more than 0.15, no more than 0.1, no more than 0.05, or no more than 0.02. Combinations of the above-referenced ranges are possible (e.g., at least 0.02 and no more than 0.9, or at least 0.1 and no more than 0.8, at least 0.9 and no more than 1), etc. Other ranges are also possible.

In one set of embodiments, a mass spectrometer comprising a tip having an opening with a cross-sectional dimension described herein (e.g., less than 100 nm, less than or equal to 65 nm, less than or equal to 60 nm, less than or equal to 50 nm, less than or equal to 40 nm, less than or equal to 30 nm, etc.) may have a mean ion transmission efficiency of at least 85% (e.g., at least 90%, at least 91%, at least 92%, at least 93%, at least 94%, at least 95%, at least 97%, at least 99%, etc.). In some embodiments, the above-referenced mean ion transmission efficiency may have a deviation of +/−3%, +/−2%, or +/−1%.

In some embodiments, tips with inner diameters less than or equal to 65 nm (e.g., less than or equal to 60 nm, less than or equal to 40 nm, less than or equal to 20 nm, etc.) may be employed to produce a high fraction (e.g., at least 0.7, at least 0.8, at least 0.9, at least 0.95, or at least 0.99, or equal to 1) of bare ions, for example, ions that do not include solvent molecules. In some embodiments, only bare ions are produced by the tips described above. In some cases, it may be particularly advantageous to emit bare ions, as opposed to ion clusters or charged droplets, as the direct emission of bare ions may allow for improved determination of different amino acids (e.g., including variants of amino acids with post-translational modifications).

In some embodiments, as the ions and/or ion cluster (if present) are emitted at the tip of the capillary into vacuum, the ions and/or ion clusters experience little, if any, of collision with a gas molecule (e.g., a background gas molecule). For example, in some embodiments, the probably of ions and/or ion clusters experiencing a collision with a gas molecule is less than about 2% (e.g., less than about 1.5%, less than about 1%, less than about 0.5%, or equals to 0%, etc.).

Certain aspects are directed to sequencing a polymer, such as a biopolymer, using an instrument comprising the ion source, for example, a mass spectrometer such as described herein.

For instance, in some embodiments, a polymer may be the species of interest. The species of interest may be a biopolymer, e.g., a protein or peptide (comprising amino acids), or a nucleic acid sequence (e.g., DNA, RNA, etc.). Other types of biopolymers, such as carbohydrates or polysaccharides, may also be used as a species of interest in some cases. In addition, it should be understood that other types of polymers may also be sequenced in some cases, e.g., artificial or synthetic polymers. Furthermore, analogously, the structures of species of interest that are not polymers may also be determined.

In some cases, for example, the structure, sequence, and/or identity of the species of interest (e.g., a polymer) can be determined by determining the ionized fragments using a detector. For example, the sequence of the species of interest can be detected by monitoring the time of arrival of individual ionized fragments (e.g., ions or ion clusters) at the detector, e.g., that are produced by ionizing the polymer as discussed above, and producing ions or ion clusters. Without wishing to be bound by any theory, it is believed that a species of interest, such as a polymer, may be ionized in substantially linear fashion, e.g., due to the size of the opening of the capillary, and the ions or ion clusters that are produced may then be determined by a detector as discussed herein, e.g., in the order in which the ions or ion clusters are produced from the species of interest. In some embodiments, the capillary comprises a carbon nanotube or a boron nitride nanotube, where the cross-sectional dimension (e.g., inner diameter) of the nanotubes is small enough, e.g., 1 nm to 2 nm, such that a polymer molecule may ionize in a sequential order that reflects the primary structure of the polymer. Of course, larger diameters, or other materials, are also possible in other embodiments, e.g., as discussed herein. It should be noted that in some cases, e.g., when the ions or ion clusters are passed into reduced pressure environments, the detector may be able to determine such ordering at relatively high fidelity, for example due to the relative lack of collisions with gas molecules as the ions or ion clusters pass through to the detector. Accordingly, based on the order at which ions or ion clusters are determined, the structure or sequence of the species of interest can be determined.

In some embodiments, the mass spectrometer described herein may comprise more than one ion source. For example, the mass spectrometer may comprise an ion source described herein, along with one or more additional ion sources. For example, in some aspects of the present disclosure, a multiplexing mass spectrometer comprising a plurality of ion sources is disclosed herein. The plurality of ion sources may be a plurality of identical (or different) ion sources. Some or all of the plurality of ion sources may comprise a capillary containing a species of interest suspended in a fluid. The multiplexing mass spectrometer may allow for simultaneous detection and sequencing of a number of species of interest contained within the capillaries of the plurality of ion sources in some embodiments.

In some embodiments, the multiplexing mass spectrometer may comprise the plurality of ion sources, a magnetic mass filter downstream the plurality of ion sources, and an array of detectors downstream of the magnetic mass filter. A non-limiting schematic representation of a multiplexing mass spectrometer is illustrated in FIG. 12. As shown, the multiplexing mass spectrometer 110 comprises a plurality of ion sources 120, a mass filter 190 (e.g., magnetic mass filter) downstream the ion sources 120, and an array of detectors 170 (e.g., imaging detectors) downstream the mass filter 190. Some or all of the plurality of ion sources illustrated in FIG. 12 may be identical to the ion source described in FIGS. 1A-1B.

For example, some or all of the ion sources may include a capillary and an electrode proximate the capillary or nanotip of the capillary. The capillary may have any properties described elsewhere herein, e.g., such as a tip portion (e.g., nanotip) with an opening, a body portion, and/or containing a species of interest suspended or dissolved in a fluid, etc. In some cases, the plurality of ion sources may be arranged in a linear array. In some cases, some or all of the plurality of capillaries may comprise nanotips arranged in a linear array. For example, as shown in FIG. 12, mass spectrometer 110 comprises a plurality of capillaries 130a, 130b, and 130c, each having a nanotip, arranged in a linear array. In some embodiments, some or all of the plurality of ion sources may contain a species of interest within the capillary. The species of interest within the ion sources may be same or different.

A multiplexing mass spectrometer may comprise any appropriate number of ion sources. For example, the multiplexing mass spectrometer may comprise at least 2 (e.g., at least 3, at least 5, at least 10, at least 25, at least 50) and/or up to 100 (e.g., up to 200, up to 500, or up to 1000) ion sources. Combination of the above-referenced ranges are possible (e.g., at least 2 and up to 1000). Other ranges are also possible.

In some embodiments, the multiplexing mass spectrometer may further comprise a light source directed towards the plurality of ion sources. The light source may have any properties and/or configurations described elsewhere herein. For example, the multiplexing mass spectrometer shown in FIG. 12 may comprise a light source (not shown) identical to the light source (e.g., UV laser) shown in FIGS. 1A-1B. As described elsewhere herein, the light source may be configured to fragments the species of interest within the capillaries (e.g., the tip portion of the capillaries) of the ion sources into individual components. The fragmented individual components may be in turn ionized from the tip of the capillary into vacuum. For example, as shown in FIG. 12, the light source may be configured to fragments the species of interest within the capillaries (e.g., the tip portion of the capillaries 130a, 130b, 130c) of the ion sources 120 into individual components, after which the individual components may be ionized into vacuum 180. The trajectories of ionized individual components from the nanotips are shown by 154.

In some embodiments, the multiplexing mass spectrometer comprises a magnetic mass filter capable of simultaneously separating the ionized individual components exiting each ion source based on their mass-to-charge ratios. The magnetic mass filter, for example, may be configured to pass the ionized fragmented individual components from each of the ion sources to the array of detectors downstream the magnetic mass filter. The array of detectors, in turn, may be configured to simultaneously detect the ionized fragmented individual components from each of the plurality of ion sources. For example, as shown in FIG. 12, a magnetic mass filter 190 (e.g., magnetic sector) may be employed to separate ionized individual components exiting each ion source based mass-to-charge ratio.

The magnetic mass filter, in accordance with some embodiments, may be employed to separate and focus the ionized individual components in both a lateral direction and a transverse direction before the individual components impinge on the array of imaging detectors. Referring to FIG. 12 as a non-limiting example, the magnetic mass filter 190 may be employed to separate and focus ionized individual components 154 in both a lateral direction and a transverse direction before the individual components 154 impinge on the array of detectors 170 (e.g., imaging detectors). The array of detectors, in accordance with some embodiments, may be arranged in a 2-dimensional array capable of detecting ionized individual components in both a lateral and a transverse direction. A multiplexing mass spectrometer having a configuration described herein may allow for simultaneously and high throughput sequencing of various types of species of interest.

The multiplexing mass spectrometer may comprise any appropriate additional components described elsewhere herein. For example, the multiplexing mass spectrometer may further comprise an ion optics (e.g., optical lens, etc.) positioned between the ion source and the mass filter. A non-limiting example of one embodiment of an ion optics is illustrated in FIGS. 1A-1B, e.g., as shown by ion optics 100.

U.S. Provisional Patent Application 63/235,601, filed Aug. 20, 2021, entitled “System and Methods for Analysis of Peptide Photodissociation for Single-Molecule Protein Sequencing.” by Stein, et al., is incorporated herein by reference in its entirety. In addition, U.S. Provisional Patent Application 63/341,992, filed May 13, 2022, “System and Methods for Analysis of Peptide Photodissociation for Single-Molecule Protein Sequencing.” by Stein, et al., is also incorporated herein by reference in its entirety, entitled “System and Methods for Analysis of Peptide Photodissociation for Single-Molecule Protein Sequencing.” by Stein, et al., is incorporated herein by reference in its entirety.

U.S. Provisional Patent Application Ser. No. 63/015,407, filed Apr. 24, 2020, entitled “Nanotip Ion Sources and Methods,” by Stein, et al., is incorporated herein by reference in its entirety.

In addition, U.S. Provisional Patent Application Ser. No. 63/179,064 filed on Apr. 23, 2021, entitled “Systems and Methods for Single-Ion Mass Spectrometry with Temporal Information.” by Stein, et al., is also incorporated herein by reference in its entirety. Int. Pat. Apl. Pub. No. PCT/US2022/025902 filed Apr. 22, 2022, entitled “Systems and Methods for Single-Ion Mass Spectrometry with Temporal Information” is also incorporated herein by reference in its entirety.

Int. Pat. Apl. Pub. No. PCT/US2021/028954, filed Apr. 23, 2021 is incorporated herein by reference in its entirety.

The following examples are intended to illustrate certain embodiments of the present disclosure, but do not exemplify the full scope of the disclosure.

### EXAMPLE 1

This example analyzes the feasibility of using light to fragment a peptide into its constituent amino acids before identifying them by mass spectrometry (MS) for the purpose of single-molecule protein sequencing. Laser power considerations strongly favor photofragmenting peptides in solution before they leave the ion source rather than in the gas phase. Ultraviolet (UV) wavelengths near 200 nm are weakly absorbed in water, and a single photon can selectively cleave the peptide bonds that link amino acids together. These properties make UV photofragmentation more promising than methods based on infrared or x-ray light. This example develops a simple model of the probability of liberating an amino acid intact by cleaving the peptide bonds on either side of it before the light damages the amino acid itself. It is predicted that 193 nm light can liberate many amino acids with probabilities ranging from 0.65-0.92; however, the aromatic amino acids and histidine, methionine, arginine, and lysine, which are relatively susceptible to photodamage, would be liberated intact with a probability in the range 0.004-0.330. These findings suggest that UV photofragmentation could reveal a significant amount of a single protein's sequence information to a mass spectrometer.

A method is described for sequencing single proteins based on the nanocapillary ion source. The basic idea is shown in FIGS. 1A-1B. A voltage applied to the source drives positively charged peptides toward the nanopore tip, which is small enough to force the peptide chain into a linear configuration. The ion source emits the constituent amino acids from liquid into vacuum in sequential order. The amino acid ions pass through a magnetic mass filter that separates them based on their mass-to-charge ratio before they impinge on an array of single-ion detectors. The location of impingement reveals the identity of an amino acid, and the timing of detections informs a reconstruction of their original sequence. One intermediate step separates individual amino acids from the parent peptide chain prior to mass filtration. Light is widely used to photofragment peptides in proteomics. This example analyzes the feasibility of using laser light to photofragment peptides in our single-molecule protein sequencing approach.

FIG. 1A is a schematic of single-molecule protein sequencing by nanopore mass spectrometry. The sketch illustrates trajectories of heavy and light amino acid ions emitted from a nanocapillary ion source. The ions pass through ion optics and a magnetic mass filter and impinge on an array of single ion detectors. An ultraviolet laser is used to fragment peptides. FIG. 1B is an illustration of an elongated peptide chain photofragmenting near the tip of the nanocapillary ion source.

To fragment a peptide before it passes through the mass filter, one can direct a laser beam in the path of the ions on the vacuum side of the ion source. The problem with this approach is that peptides would pass through the beam extremely quickly. Consider, for example, an arginine dipeptide, which has a mass of 330 amu and a charge of +2e. If an extraction voltage of 300 V is applied (near the lower bound required by the ion source), the dipeptide ion would acquire a kinetic energy of 600 eV and travel the full distance from the source to the detector of our instrument within less than 20 microseconds; it would spend even less time in the path of the beam. This sets a lower limit on the irradiation power necessary to achieve a high-probability of fragmentation. For comparison, in previous studies a 50 W CO₂ laser irradiated peptides for 10 ms or longer to induce photo-fragmentation in vacuum. To deliver the same amount of energy in 20 microseconds as a 50 W laser does in 10 ms would require a laser power of 25,000 W. Thus, an impractically powerful laser would be required to fragment peptides on the vacuum side of the ion source.

Another possibility is to direct the laser at peptides while they are still in solution inside the nanocapillary ion source. There, peptides travel at least seven orders of magnitude more slowly than in vacuum. The fastest transport process appears to be an electro-convective flow mechanism.

The flow of ions along the charged surfaces of a Taylor cone induces circulating fluid flows that reach maximum speeds near the tip. Top speeds are estimated on the order of 10⁻⁴ m/s in the nanocapillaries. Another relevant transport mechanism is electrophoresis; however, even the greatest electric fields in the instrument in this example acting on the most electrophoretically mobile peptides give migration speeds that are an order of magnitude slower than the electro-convective flow. Brownian motion is also a relatively slow transport process over distances comparable with the radius of a laser beam. The most diffusive peptides would take almost 3 minutes to diffuse a distance of 0.5 mm-two orders of magnitude longer than the electro-convective flow would take to travel the same distance. Thus, peptides in liquid should transit a laser beam slowly enough that a 0.1 W laser would deliver the same energy to them as a 50 W laser does in 10 ms. Evidently, targeting peptides while they are still in solution reduces the demands on the incident laser power sufficiently that liquid photofragmentation is feasible and safe.

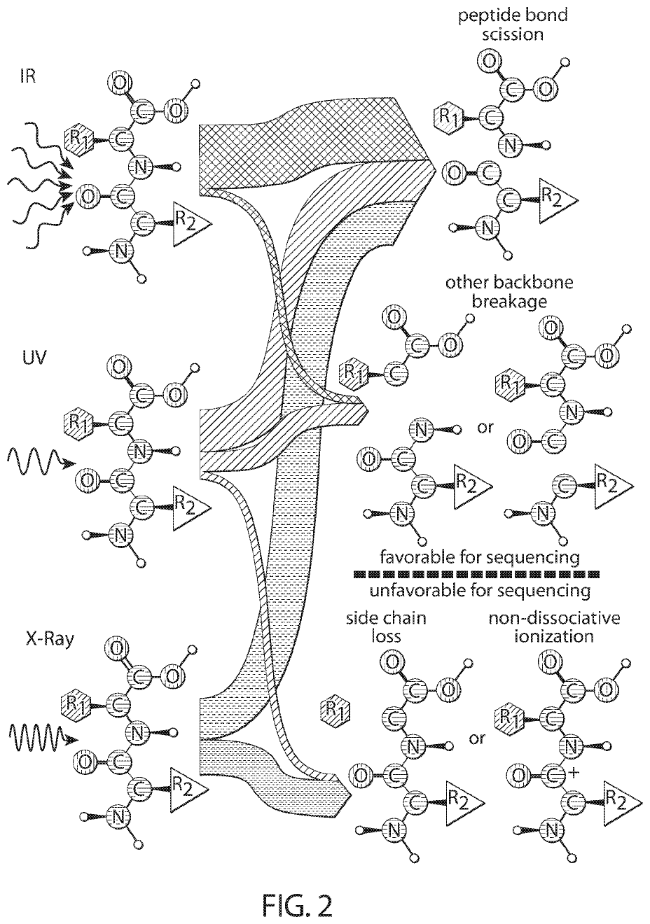

The wavelength of light influences the types of molecular fragments that result from photofragmentation. FIG. 2 illustrates the structure of a dipeptide, the structures of common photofragmentation products, and the approximate frequency with which laser light in different wavelength regimes induces particular transformations in vacuum. The parent dipeptide comprises two amino acids that share a chemical backbone. The backbone's peptide bond requires only approximately 4 eV to break and is among the molecule's most labile bonds. If one could break that bond selectively, it should be straightforward to identify the resulting amino acids by their mass. Alternatively, breaking a different bond in the backbone would shift some mass from one amino acid to its neighbor, but one should be able to account for that mass shift and still identify each amino acid. However, if photons damage or eject the amino acid side chains, which are the distinguishing features, this could significantly complicate our protein sequencing scheme.

FIG. 2 shows a comparison of peptide photofragmentation by IR, UV, and soft X-ray light. Line thicknesses indicate relative abundances of significant fragmentation products, as approximated from studies in vacuum.

The absorption of light by water further constrains the properties of the light that can be used for sequencing. The linear absorption coefficient of water (μ) at 10.6 micrometers is 10⁵ m⁻¹, so an incident IR laser beam would be absorbed over a characteristic distance of only 10 μm 35. This suggests that a very small fraction of laser power would be absorbed by peptides in solution, and that the incident power required to induce multiphoton dissociation processes may be prohibitively high. The absorption of ultraviolet light in water is orders of magnitude weaker, with μ=10 m⁻¹ at 193 nm and μ=1 m⁻¹ at 222 nm, giving characteristic absorption distances of 10 cm and 1 m, respectively. The heating effect probably sets the upper limit on the incident power density at a given wavelength. It is believed that if the absorbed light heats the water in the nanocapillary to the boiling point, it would likely interfere with the ion evaporation process.

It was numerically calculated that the temperature rise that should result from the absorption of different wavelengths of light in the IR and UV. A capillary was modeled as a truncated cone with a tip radius of 20 nm, a length of 500 micrometers, and a cone aperture of 6°. It was assumed the cone is irradiated from the side (the light arrives perpendicular to the cone axis) by a 1 mm wide laser beam centered at the capillary tip; this leaves the entire cone exposed to a uniform incident power density, ρ. For example, a 1 W laser beam focused to a diameter of 1 mm produces a power density of 1.3×10⁶ W/m². The steady-state temperature distribution was calculated by solving the steady-state heat equation

$$k {∇}^{2} T ( \vec{r} ) = - {\dot{q}}_{v} ( \vec{r} ) , \qquad ( 1 )$$

where k=0.6 Wm⁻¹K⁻¹ is the thermal conductivity of water at 300 K. T(r) is the temperature at a position r, and qv(r) is the volumetric heat generation (Wm⁻³) caused by light absorption. To simplify the calculations, it was assumed that qv(r) depends only on the axial distance from the tip of the cone, z, and it was obtained that qv(z) by computing the mean power density absorbed by each thin circular cross section at axial position z, ignoring the refraction of light entering the cone, but accounting for u as rays of light penetrated to different depths. Zero heat flux boundary conditions were imposed at the tip and the sides of the cone, and held the base at a constant temperature. Eq. 1 was solved for an axisymmetric cone by finite element analysis of a two-dimensional mesh using the Partial Differential Equations Toolbox in Matlab.

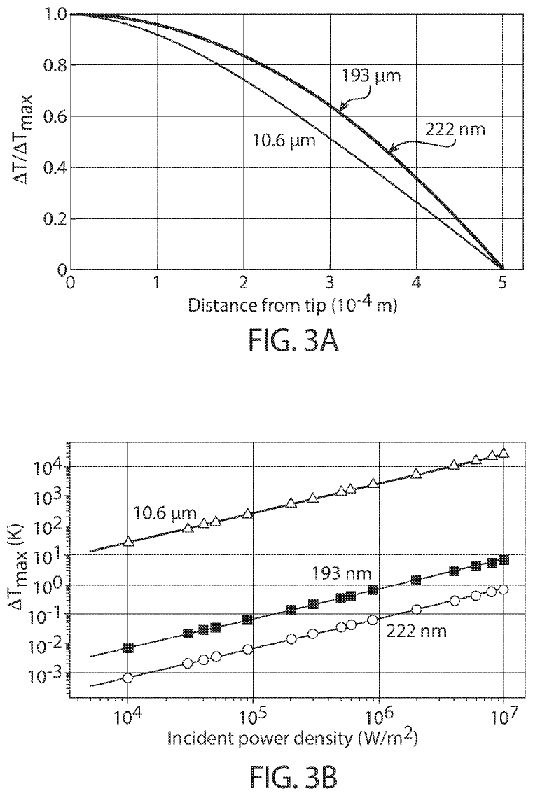

FIG. 3A shows the normalized, steady-state temperature distributions along the axis of the cone. The temperature rises monotonically from the base to the tip for all wavelengths studied, with a relatively steeper rise for the UV wavelengths 193 nm and 222 nm than for the IR wavelength 10.6 micrometers.

FIG. 3B plots the maximum temperature rise at the tip as a function of ρ. It depends strongly on wavelength. 10.6 micrometer light causes significant heating at the tip, with a power density of only about 4×10⁴ W/m²—equivalent to a 32 mW laser with a 1 mm beam diameter—needed to reach the boiling point of water in a room temperature experiment. The UV wavelengths cause minimal heating, by contrast; even with an extremely high power density of 10⁷ W/m², 193 nm and 222 nm light should heat the tip by less than 10 K and 1 K, respectively. These results indicate that the heating of water limits the use of IR light in this sequencing scheme.

FIG. 3A shows calculated heating profile in a nanocapillary under steady irradiation by 10.6 μm. 193 nm, and 222 nm light. FIG. 3B shows dependence of the maximum temperature increase in a nanocapillary on the incident laser power density for 10.6 micrometers (triangles), 193 nm (squares), and 222 nm (dots) light. Symbols show results of finite element calculations, and curves are linear fits to the data.

To summarize this comparison of wavelengths, UV light offers relatively specific cleavage of peptide backbones, low power requirements, and low absorption by water, all of which are favorable for sequencing single proteins.

This example now estimates whether UV light can reliably separate the amino acids from one another without damaging them to the point that they cannot be identified. The probability of cleaving the backbone between two amino acids should increase with UV exposure, but so should the likelihood of cleaving other bonds, which may complicate sequencing. To evaluate those tradeoffs, a simple, probabilistic model of the photofragmentation processes was developed.

Table 1 shows UV absorption and photodecomposition properties of peptides and amino acids.

TABLE 1

| Absorber | εᵢ (cm⁻¹M⁻¹) | σᵢ · 10²¹ (m²) | Φᵢ (%) |
|---|---|---|---|
| peptide bond | 6500 | 2.5 | 5.9 |
| Tyr | 43000 | 16.4 | 9.3 |
| Phe | 28100 | 10.7 | 5.7 |
| Trp | 2\|0000 | 7.6 | 6.7 |
| His | 5100 | 2.0 | 26.0 |
| Met | 2300 | 0.8 | 26.2 |
| Arg | 11200 | 4.3 | 1.8 |
| Lys | 1100 | 0.4 | 14.5 |
| Val | 260 | 0.1 | 14.5 |
| Thr | 350 | 0.1 | 10.5 |
| Leu | 250 | 0.1 | 13.9 |
| Ser | 290 | 0.1 | 10.3 |
| Ala | 280 | 0.1 | 8.2 |
| Pro | 290 | 0.1 | 6.7 |
| Hyp | 280 | 0.1 | 5.1 |
| Gly | 240 | 0.1 | 5.9 |
| Asp | 240 | 0.1 | 2.0 |

Peptide bonds and amino acids behave as independent UV absorbers in aqueous solution. The molar absorption coefficient, εᵢ, is attributable to each one at 193 nm. Consequently, a particular peptide bond or amino acid absorbs photons at an average rate Joi, where J is the local photon flux and σᵢ is the absorption cross section of absorber i that is calculated from εᵢ. Following the absorption of a photon, the added energy may either lead to fragmentation of the molecule or to vibrational dissipation processes that leave the chemical bonds intact. The fraction of absorbed photons that cause photodissociation, Φᵢ is also called the quantum efficiency. Combining the absorption and dissociation processes leads to the average photodissociation rate for a
particular species, JσᵢΦᵢ. The cumulative probability that a particular peptide or amino acid has undergone photo-dissociation, P_{d,i}, grows with time t according to

$${P}_{d , i} = 1 - {e}^{- J {σ}_{i} {Φ}_{i} t} . \qquad ( 2 )$$

Experimental values of ε, σᵢ, and Φᵢ for fifteen of the twenty different amino acids and the peptide bond, as well as for hydroxyproline (a common, modified form of the amino acid proline) are compiled in Table 1. Experimental values for the amino acids asparagine (asn), cysteine (cys), glutamine (gln), glutamic acid (glu), and isoleucine (ile) are not available.

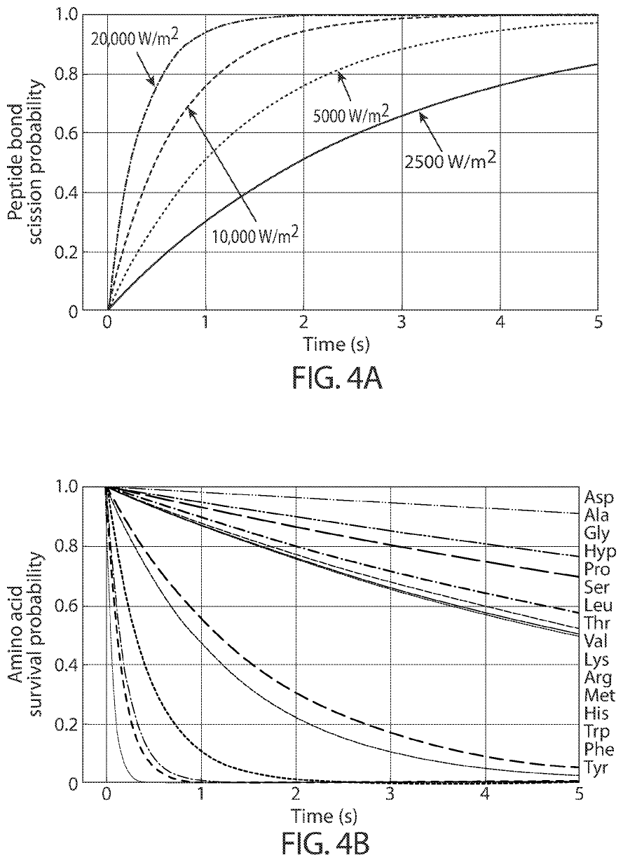

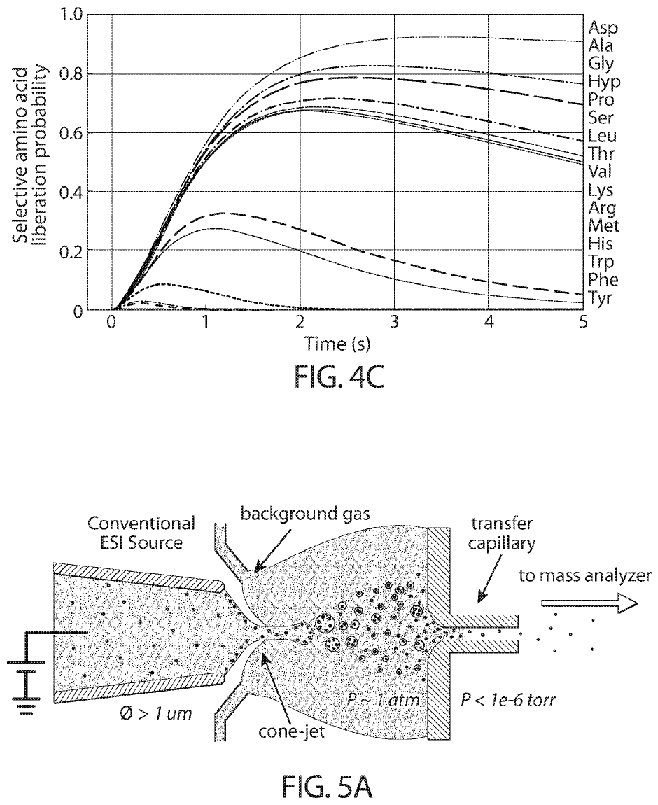

FIG. 4A compares the cumulative probability of peptide bond dissociation, P_{d,pep}, for different values of ρ. P_{d,pep} rises and asymptotically approaches unity on a characteristic timescale that is inversely related to ρ. The characteristic timescale is 0.7 s for ρ=10, 000 W/m², a power density which roughly corresponds to a 10 mW, 1 mm wide laser beam.

It may also be useful to estimate number of amino acids that can survive exposure to the UV light without undergoing photodissociating. The cumulative survival probability of amino acid i, P^{s,i}, is

$${P}_{s , i} = 1 - {P}_{d , i} = {e}^{- J {σ}_{i} {Φ}_{i} t} . \qquad ( 3 )$$

FIG. 4B plots P_{s,i} for 16 different amino acids for r=10,000 W/m². An amino acid's ability to survive, as measured by the characteristic decomposition time (JσᵢΦᵢ)⁻¹, depends strongly its type. The aromatic amino acids tyrosine (Tyr), phenylalanine (Phe), and tryptophan (Trp) decay relatively quickly, undergoing photodecomposition on timescales of 0.07 s. 0.17 s, and 0.20 s, respectively. Histidine (His) also decays relatively quickly, with a characteristic decomposition time of 0.20 s. By comparison, the amino acids valine (Val), threonine (Thr), leucine (Leu), serine (Ser), proline (Pro), hydroxyproline (Hyp), glycine (Gly), alanine (Ala), and aspartic acid (Asp) are long-lived, with decom-position timescales in the range 7-56 s. The decomposition time of methionine (Met), arginine (Arg), and lysine (Lys) lies between the long- and short-lived groups.

This example also examines the probability of liberating a given amino acid from a protein without damaging the amino acid beyond recognition. The most straightforward mechanism is to fragment the two peptide bonds that link amino acid i to the peptide chain without inducing photodissociation of the amino acid itself; the cumulative probability of such selective cleaving, P_{sel,i}, is obtained by combining Eqs. 2 and 3:

$${P}_{sel , i} = {{P}_{s , i} ( {P}_{d , pep} )}^{2} . \qquad ( 4 )$$

FIG. 4C plots the time evolution of P_{sel,i} for 16 amino acids with ρ=10, 000 W/m². In all cases, P_{sel,i} grows with time before reaching a peak and subsequently decaying. The peak is higher and occurs after longer UV exposures for the amino acids with longer characteristic dissociation times. P_{sel,i} reaches a peak in the range 0.004-0.028 and within 0.25 s for the aromatic amino acids and His. The peak for the long-lived amino acids is in the range 0.65-0.92 after exposure times in the range 2 s-3 s. The intermediate group (Met, Arg, and Lys) reaches a peak P_{sel,i} in the range 0.086-1.33 after exposure times in the range 0.5 s-1.5 s.

FIG. 4A shows cumulative probability of peptide bond dissociation, obtained from Eq. 2 for exposure to 193 nm laser light of different intensities, as indicated. FIG. 4B shows the probability of amino acids not decomposing as a function of exposure time to 193 nm laser light with ρ=10,000 Wm⁻², calculated according to Eq. 3. FIG. 4C shows the probability of selective amino acid liberation (i.e., fragmenting the two peptide bonds joining an amino acid to a peptide without damaging the amino acid) as a function of exposure time to 193 nm laser light with ρ=10,000 Wm⁻². Probability calculated according to Eq. 4. In FIGS. 4B and 4C, the different amino acids are indicated by color and ordered by probability.

One finding is that it should be possible for UV light to completely liberate undamaged amino acids from a protein with a reasonably high efficiency. Liberated amino acids are defined as those resulting from the scission of the two flanking peptide bonds. Many amino acids can be liberated with a probability in the range 65-92%. If the possibility of liberating amino acids by cleaving different (i.e., non-peptide) bonds is also considered along the backbone, or that an amino acid may still be identifiable by its mass after other photodissociation processes, the fraction of identifiable fragments should increase. For example, breaking a bond in an aromatic group of an amino acid may significantly change its photoabsorption spectrum (a change that would be recognized as photodissociation in optical measurements) without altering its mass.

In conclusion, UV light offers a promising route to fragmenting peptides into their constituent amino acids for single-molecule analysis. The extremely high speeds with which ions travel from the ion source to the detector in vacuum make it preferable to photofragment peptides while they are in solution, prior to the extraction of ions. Ultraviolet wavelengths near 200 nm are the most promising for sequencing thanks to their low absorption in water, the relatively high selectivity with which they fragment peptide backbone bonds, and the modest laser power requirements that a single-photon bond-cleavage process entails. The calculations of the rates of competing photochemical processes indicate that it should be possible to cleave the peptide bonds flanking many amino acids before they undergo damage to the identifying side chain. The accuracy of amino acid calls in sequencing can exceed 90% for the most stable side chains, but is expected to fall for increasingly labile side chains. Future measurements of the photofragmentation products and the relative selectivity of different wavelengths could be used to optimize UV photofragmentation for analysis of the composition and sequence of a single protein.

### EXAMPLE 2

### Introduction

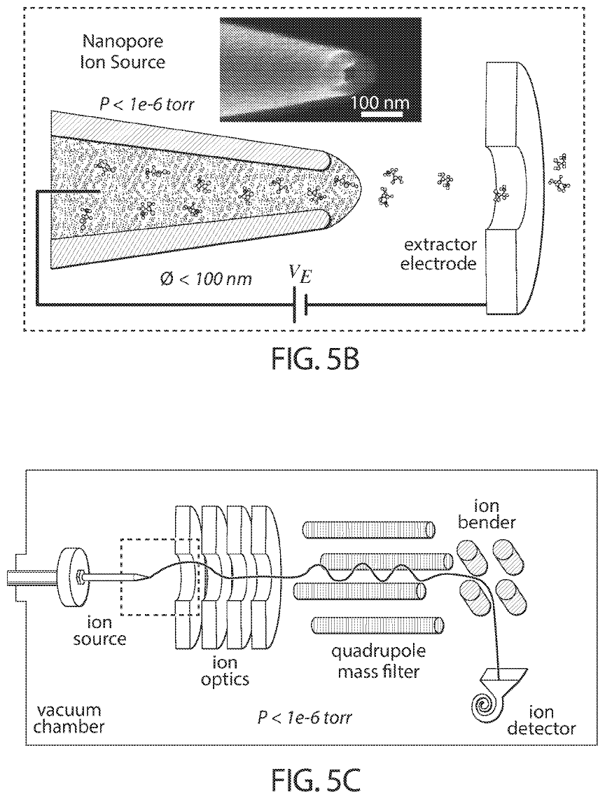

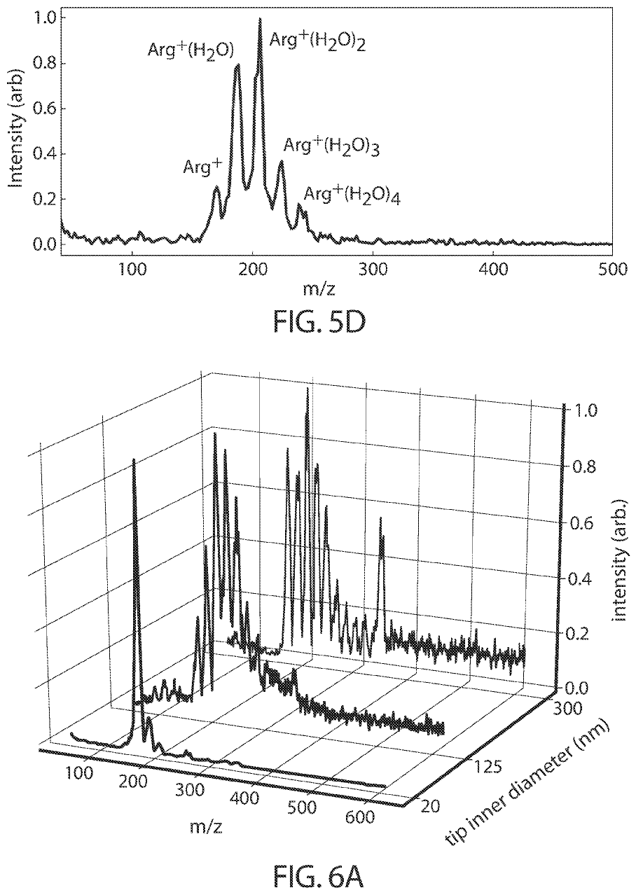

Mass spectrometry (MS) is the workhorse of proteomics research thanks to its ability to distinguish amino acids and small peptides by their mass. Its utility also significantly derives from the availability of soft ionization techniques for transferring peptide ions into the gas phase intact. In particular, electrospray ionization (ESI) transfers analyte into a mass spectrometer via a plume of charged droplets that emerge from a liquid cone-jet at the end of a voltage-biased capillary, as illustrated in FIG. 5A. The droplets pass through a background gas that induces a string of evaporation and Coulomb explosion cycles that ultimately release analyte ions into the gas phase. However, the background gas needed to liberate ions from droplets is also a source of significant sample loss that limits the sensitivity of MS.

The background gas and the plume of charged droplets it creates widely disperse ions, the majority of which collide with the transfer capillary, which bridges the ambient-pressure ion source and the first pumping stage of the mass spectrometer, or other hardware components upstream of the detector. Early ESI sources had emitter tips with diameters of hundreds of micrometers, and only one ion in ˜10⁴ reached the mass analyzer. Nano-electrospray ionization (nano-ESI) increased the ion transmission efficiency to ˜1% in typical measurements (occasionally reaching as high as 12%) by using emitters with micrometer-scale tips that reduced the flow rates to the range of several nL/min. However, co-optimizing the efficiency of multiple analytes is fundamentally challenging because ESI involves processes that physically separate different ion species within the plume. State-of-the-art MS instrumentation still requires thousands to millions of copies of proteins for their identification. This sensitivity falls short of what is desired for single-cell proteomics and single molecule analyses. Achieving single-molecule sensitivity requires an ion source that circumvents the loss mechanisms from spraying charged droplets into a background gas.

Presented herein in this example is a nanopore ion source that emits amino acid and small peptide ions directly into high vacuum from its tip (FIG. 5B). The ion source included a pulled quartz capillary with a tip whose inner tip diameter is smaller than 100 nm. It is reasoned that the smallness of the tip can potentially influence ion emission in several ways: First, the surface tension of water can maintain a stable liquid-vacuum interface that supports many atmospheres of pressure when stretched across a nanoscale opening. Second, the fluid flow rate, which scales as the inverse cube of the tip diameter, may be too low for a stable electrospray cone-jet to form, and this may prevent charged droplets from being emitted altogether. Third, electric fields may concentrate at a sharp, conductive tip like an electrolyte-filled nanocapillary, reach ˜1 V/nm at the meniscus, and draw out ions at high rates by the process of ion evaporation.

Described herein in this example is the characterization of the emission of ions by a nanopore ion source from aqueous solution directly into high vacuum. Mass spectra of amino acids and small peptides were obtained using a custom quadrupole mass spectrometer in which the nanopore ion source operated at pressures below 10⁻⁶ torr (FIG. 5C). Separately, greater than 93% transmission of current was measured between an electrolyte filled ion source and a downstream Faraday cup. Further, the contributions of charged droplets and ions to the tip current were separated using a magnetic sector and thereby demonstrated that the nanopore ion source can be made to emit only ions. This example demonstrates the simplicity with which the nanopore ion source described herein efficiently transfers ions into high vacuum without the complications of conventional ESI, such as ion funnels, multiple pumping stages, transfer capillaries, and droplet plumes.

### Results

Emitting Amino Acid Ions from a Nanopore Ion Source

The emission of amino acids from aqueous solution were characterized in a custom quadrupole mass spectrometer shown in FIG. 5C. In a typical experiment, ion emission from a nanopore ion source was initiated by applying an extraction voltage, V_{E}, in the range +260 V to +360 V between the tip and the extraction electrode. This range of V_{E} may be significantly lower than the voltages typically needed to initiate electrospray in conventional ESI or nano-ESI sources. The tip current, I_{T}, employed was typically in the range of 3-20 pA. The onset of current can be abrupt and usually accompanied by the measurement of ions striking the instrument's detector. Easily interpretable mass spectra were collected within minutes at these low tip currents.

FIG. 5D shows the mass spectrum of a 100 mM solution of arginine in water. This spectrum was obtained in positive ion mode using a nanopore ion source with inner tip diameter of 41 nm. Five peaks are clearly visible. The peak at 175 m/z corresponds to the singly charged arginine ion (Arg⁺). The higher m/z peaks are all separated by 18 m/z, the shift induced by an additional water molecule. Thus, the other peaks correspond to solvated states of arginine (Arg⁺(H₂O)ₙ), where the solvation number n ranges from 1 to 4.

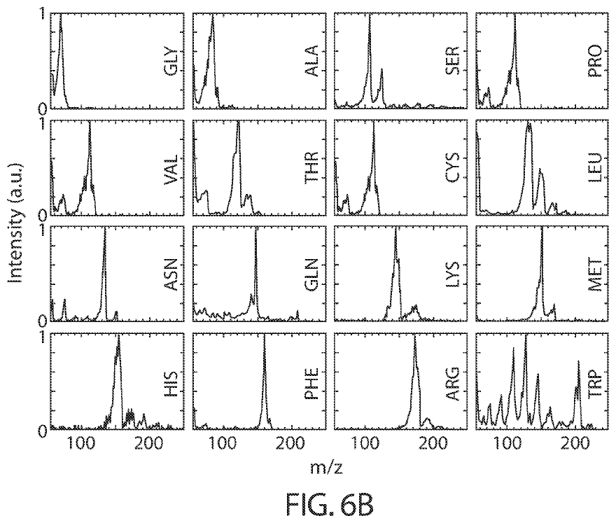

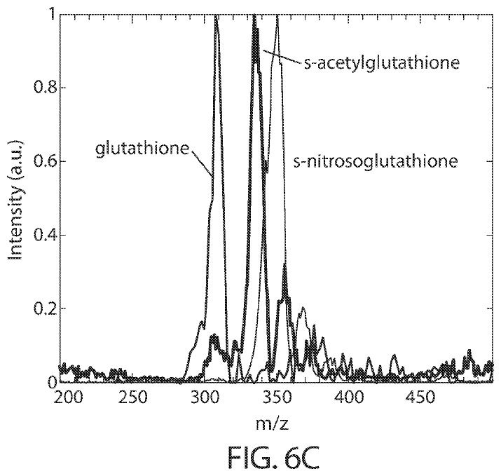

FIG. 6A illustrates how the tip diameter influenced arginine mass spectra. The spectra shown were obtained using nanocapillaries with inner tip diameters of 20 nm, 125 nm, and 300 nm. The largest tip produced a broad spectrum of peaks that included the bare arginine ion, eight incrementally hydrated arginine ion clusters, and a peak at 349 m/z that corresponded to the arginine dimer ion (Arg·Arg+H)⁺. The intermediate-sized tip produced a narrower spectrum that included the bare arginine ion, six incrementally hydrated arginine ion clusters, and a relatively diminished arginine dimer ion peak. The smallest tip primarily produced the bare arginine ion, but attenuated peaks corresponding to the singly and doubly hydrated arginine ion clusters were also visible in the spectrum. Smaller tips tended to produce relatively stronger signals and less noisy spectra than larger tips, as can be seen by comparing the baselines of the three spectra in FIG. 6A. Some variance was observed in the distribution of solvation states between nanocapillaries with similar tip sizes (e.g., when comparing the spectrum in FIG. 5D produced by a 41 nm tip to the spectrum from FIG. 6A produced by a 20 nm tip). However, only nanocapillaries with inner tip diameters smaller than about 65 nm have produced spectra where most of the amino acid ions were measured in the unsolvated state.

FIG. 6B shows mass spectra obtained from 16 different aqueous amino acid solutions, all at 100 mM concentration with the exception of tryptophan which was at 50 mM. Four different nanopocapillaries with inner tip diameters of 20, 25, 57 and 58 nm were used for these measurements. The most prominent amino acid peak in every spectrum shown in FIG. 6B corresponded to a singly charged and unsolvated ion. The spectra for glycine, alanine, proline, valine, cysteine, glutamine, and phenylalanine showed no additional peaks which could correspond to solvated amino acid ions. The spectra for serine, threonine, asparagine, lysine, methionine, histidine, arginine, and tryptophan showed a secondary peak 18 m/z to the right of the unsolvated peak, corresponding to the singly hydrated amino acid ion. Leucine showed a third and possibly fourth peak corresponding to higher solvation states. The tryptophan spectrum showed peaks below 200 m/z that were consistent with hydrated states of the hydronium ion and that also appeared in control measurements of aqueous solutions with no amino acid present. Tryptophan, which has a lower solubility than the other amino acids studied, produced a relatively weak signal. Four proteinogenic amino acids were absent from FIG. 6B: no attempt to measure aspartic acid and glutamic acid was made in positive ion mode because of their low isoelectric points; isoleucine was also ignored because it was indistinguishable from leucine based on m/z, and tyrosine gave poor emission characteristics, likely related to its low solubility in water.

### Measuring Post-Translationally Modified Peptides

FIG. 6C shows mass spectra of glutathione and two chemically modified variants, s-nitrosoglutathione, and s-acetylglutathione. Glutathione is a tripeptide found in high concentrations in most cells, and the variants studied herein result from common post-translational modifications. Ion sources with 20 nm inner diameter tips generated the peptide ions from 100 mM aqueous solutions with a pH between 3.1 and 3.9, adjusted by the addition of acetic acid. The glutathione spectrum shows a single peak at 307 m/z, which corresponds to the singly charged, unsolvated glutathione ion. The spectra of s-acetylglutathione and s-nitrosoglutathione show dominant peaks at 349 m/z and 336 m/z, respectively, corresponding to the singly charged, unsolvated peptide ions; each spectrum also shows two progressively smaller peaks 18 and 36 m/z to the right of the dominant peak, corresponding to singly and doubly solvated peptide ions, respectively.

### Ion Transmission Efficiency

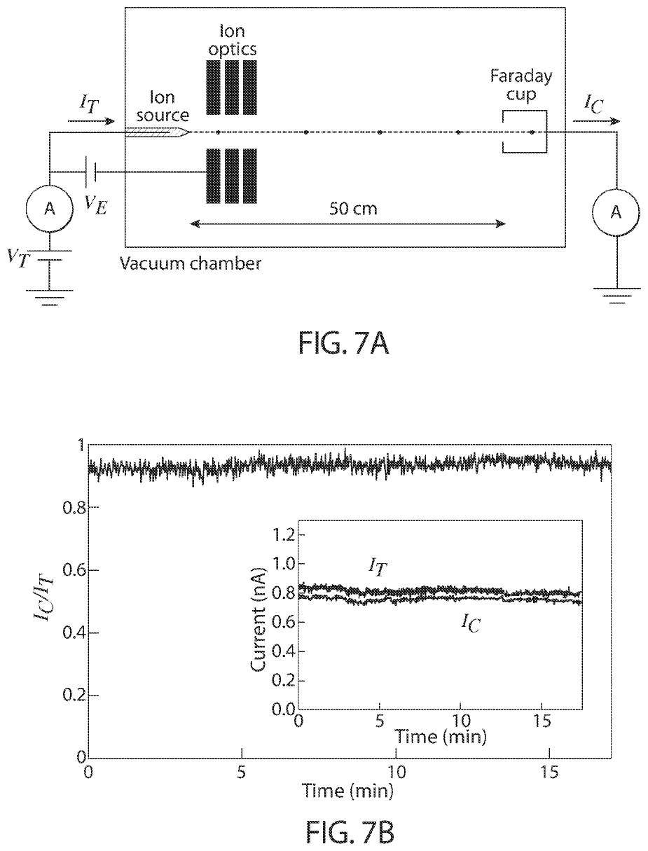

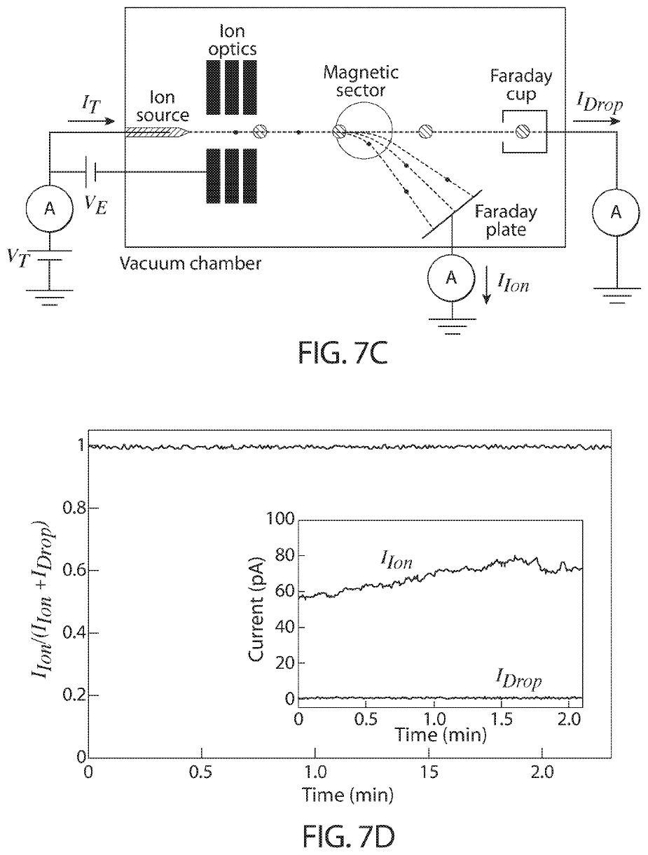

The efficiency with which ions pass from the nanopore source to a distant detector in a high vacuum environment was measured (FIG. 7A). Ions emitted by the source were focused into the 2 cm opening of a Faraday cup located ˜50 cm away. The ratio of the current collected by the Faraday cup, I_{C}, to I_{T} was the ion transmission efficiency. FIG. 7B shows I_{C}, I_{T}, and the ion transmission efficiency measured over the course of a 17 minute-long experiment using a tip with a 39 nm inner diameter filled with a 100 mM aqueous solution of sodium iodide. The mean ion transmission efficiency measured was 93.4%+/−1.7%. Although I_{T} drifts between about 780 pA and 840 pA on a timescale of minutes, the slow rises and falls in I_{T} are mirrored by I_{C}, resulting in a relatively stable transmission efficiency.

### Separating Ions and Charged Droplets

The possibility that the nanopore source emits charged droplets in addition to ions was investigated by adding a magnetic sector to the flight path as shown in FIG. 7C. The 6 cm diameter, 0.54 T magnetic sector deflected charged species based on their mass-to-charge ratio. Droplets larger than 15 nm in diameter were deflected by less than 2.7° and entered the Faraday cup, even if they were charged to the Rayleigh limit. The Faraday cup was used to measure the current from charged droplets, I_{Drop}. Meanwhile, ions with m/z in the range ˜100 to ˜350 were deflected onto a separate Faraday plate and produced an ion current/Ion. FIG. 7D shows I_{Ion}, I_{Drop}, and the ionic fraction of the total measured current

$$( \frac{{I}_{Ion}}{{I}_{Ion} + {I}_{Drop}} )$$

for a 2 minute-long measurement performed using a 28 nm tip filled with a 100 mM aqueous solution of NaI·/I_{Ion} raised from about 60 pA to about 80 pA while no/Drop was observed. The nanopore source appeared to emit only ions; however, this measurement cannot exclude the occurrence of highly charged droplets smaller than about 15 nm.

### Calculating the Probability of Ions Scattering

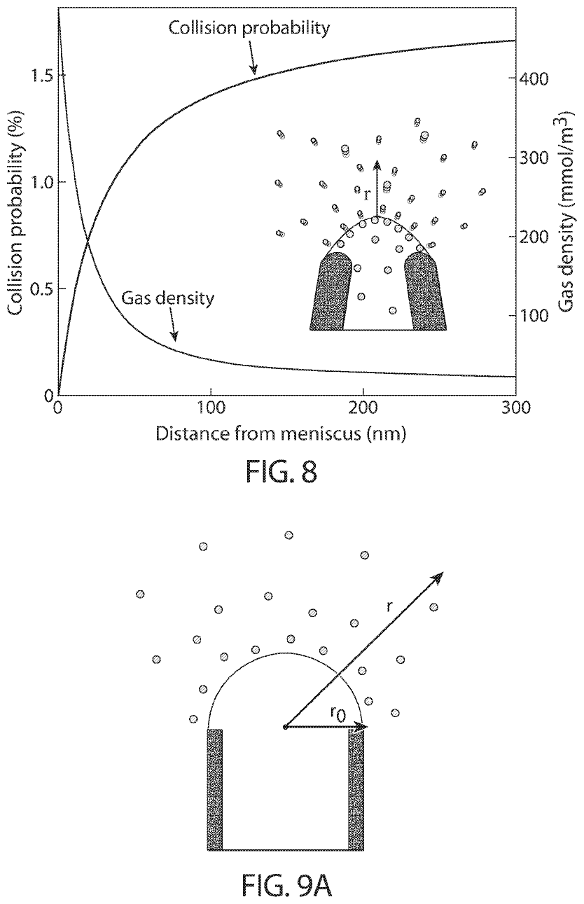

Calculations indicated that most ions traced collisionless trajectories from the ion source to the detector. FIG. 8 shows the probability of an amino acid ion dressed with a hydration shell colliding with a gas molecule in this example, based on the kinetic theory of gases. It was assumed that ions pass through a distribution of evaporating water molecules and a homogeneous low-pressure background of N₂. FIG. 8 sketches the physical situation and plots the number density of gas molecules and the cumulative collision probability as functions of the distance from the meniscus. The cumulative probability of an ion colliding with a gas molecule over the entire 50 cm trajectory from the source to the detector was just 2.1%, suggesting that the vast majority of ions did not experience any collisions. Most of the collisions occurred within 200 nm of the liquid meniscus due to the high density of evaporated water molecules there. A detailed description of these calculations is found in the Supplemental Information, below.

### Discussion

Two lines of evidence led to ruling out the conventional, droplet-mediated electrospray mechanism (FIG. 5A) as the main source of ions that were measured. First, no droplets larger than 10 nm were measured among the charged species delivered by the source (FIG. 7D). Second, the instrument lacked the background gas that normally sustains the evaporation of water from droplets in an electrospray. In high vacuum, nanoscale aqueous droplets shed only a small fraction of their mass before the evaporation process freezes up due to latent heat loss. Therefore, a sustained release of ions from droplets cannot occur in this instrument.

These findings can be explained with an alternative ion emission mechanism: the evaporation of ions directly from the liquid-vacuum interface at the nanopore, as illustrated in FIG. 5B. Ion evaporation is a thermal process by which ions escape from a liquid with assistance from a strong electric field at the surface. A strong enough electric field typically arises when the ratio of the conductivity, K, to the flow rate, Q, of an electrified liquid is sufficiently large. Accordingly, previous studies have observed ion evaporation from highly conductive liquids such as liquid metals, ionic liquids, and concentrated electrolyte solutions in formamide. The amino acid solutions that were measured have relatively low conductivities (in the range 0.01-0.5 S/m), but the flow rate produced in a nanocapillary under 1 atm of applied pressure is exceptionally low (<10 pL/min), so the resulting K/Q was still large. K/Q values for this Example are of the same order as those reported for the ionic liquid EMI-BF₄ while exhibiting ion evaporation with no droplet emission. Furthermore, the distributions of ion solvation states that were measured in FIG. 5D and elsewhere were similar to the distributions measured by from sodium iodide in formamide, which was also attributed to ion evaporation.

It was observed that mostly bare ions, rather than hydrated ion clusters, were measured in FIGS. 6B-6C. Experiments described herein were performed to determine whether ions were i) emitted in bare state or ii) were emitted in a hydrated state and subsequently shed their hydration shells on the way to the detector. Because only about 2% of emitted ions would experience even a single collision (FIG. 8), collisions with gas molecules were ruled out as a mechanism for desolvating ion clusters. Furthermore, the tip size appeared to influence the hydration state (FIG. 6A); this suggested that it was the local environment at the source that controlled the hydration state rather than processes occurring in flight.

The high ion transmission efficiency (FIG. 7B) was a direct consequence of the nanopore ion source's emission mechanism. Ion evaporation allowed individual ions to transfer directly into a high vacuum environment where they neither collide with background gas molecules nor undergo coulomb explosions that would propel charged species in random directions. The trajectory of each ion emitted from the source was mainly determined by the electric fields that were shaped by the ion optics.

In summary, demonstrated herein is a nanopore ion source that can emit amino acid and small peptide ions directly into high vacuum. The ability to emit bare ions, as opposed to solvated ion clusters or charged droplets, facilitated the identification of different amino acids and post-translational modifications. Ions evidently evaporated directly from the liquid meniscus at the tip, which removed the need for a background gas to free ions from droplets. By doing away with background gas collisions and the need to transfer ions from ambient pressure to high vacuum, the nanopore ion source was able to remove the primary modes of ion loss which are characteristic of electrospray ionization.

### Methods

### Preparing Nanocapillaries

Nanocapillaries were pulled from 7.5 cm long quartz capillaries with 0.7 mm inner diameter and 1 mm outer diameter (QF100-70-7.5 from Sutter Instruments). A laser puller (P-2000, Sutter Instruments) pulled nanocapillaries with sub-100 nm tips according to the following single-line recipe: heat=650, velocity=45, delay=175, pull=190. The nanocapillaries were coated with 5 nm of carbon and imaged by scanning electron microscopy (LEO 1530 VP, Zeiss) to measure the tip size. The nanocapillaries were plasma cleaned in air using a plasma preen (Plasmatic Systems Inc.) for two minutes prior to being filled with analyte solution.

### Amino Acid Solutions

Amino acid solutions were prepared by dissolving the amino acid of interest (Sigma-Aldrich) in DI water (Millipore) at concentrations of 100 mM, with the exception of tryptophan which was prepared at a concentration of 50 mM. Between 0.1-0.5% v/v glacial acetic acid (Sigma-Aldrich) was added to the amino acid solutions to reduce the pH below the amino acid's isoelectric point. Glutathione, s-acetylglutathione, and s-nitrosoglutathione solutions were prepared by dissolving the peptides in DI water at concentrations of 100 mM. The glutathione and s-acetylglutathione were purchased in powder form (Sigma-Aldrich), and the S-nitrosoglutathione was lab synthesized from glutathione according to a protocol from T.W. Hart. The pH and conductivities of each solution were measured using a pH meter (Ultrabasic Benchtop, Denver Instruments) and conductivity meter (Sension+ EC71 GLP, Hach) respectively.

### Delivering Solutions to the Ion Source

Sample solutions were delivered to the nanocapillary tip and flushed away by a tube-in-tube system. A thin inner PEEK tube (150 micrometer i.d., 360 micrometer o.d.) (IDEX Health and Science) carried sample solution in while a wider PEEK tube (0.04 i.d., 1/16″ o.d.) (IDEX Health and Science) carried used solution away from the tip around the outside of the inner tube. A syringe pump (NE-300, New Era Pump Systems) was used to supply fresh solution to the ion source view the inner tube. A VacuTight upchurch fitting (IDEX Health and Science) was used to create a seal around the base of the nanocapillary and end of the outer tube to prevent the solution from leaking into the vacuum. The tube-in-tube system was housed in a ¼ inch diameter steel tube which entered the vacuum chamber of the mass spectrometer through a KF-40 to Quick-Connect adapter (Lesker Vacuum).

### Quadrupole Mass Spectrometer

The instrument used for all the amino acid and peptide measurements presented in this Example is a custom-built quadrupole mass spectrometer. The instrument comprises a custom einzel lens, a quadrupole mass filter (MAX-500, Extrel), an ion bender (Extrel) and a channel electron multiplier detector with a conversion dynode (DeTech 413), which is sensitive to single ions. The base pressure of the instrument is around 10⁻⁸ torr. When a nanopore ion source is introduced into the mass spectrometer the pressure typically rises to 10⁻⁷-10⁻⁶ torr.

### Amino Acid and Glutathione Measurements

Nanocapillaries were prefilled with the amino acid or peptide solution using a microfil flexible needle (World Precision Instruments). The filled nanocapillaries were then mounted on the tube-in-tube system, and inserted into the mass spectrometer. The solution at the tip was continuously refreshed by pumping solution through the inner tubing from a syringe pump (NE-300, New Era Pump Systems) at a rate of 0.4 mL/hour. A voltage of +100 V was applied to the electrode inside the capillary using a high voltage source-meter (2657A, Keithley Instruments) while a negative voltage was slowly applied to the extraction electrode using a high voltage power supply (Burle) until ionization was observed. The onset of emission typically occurred when the total extraction voltage was between 200 and 350 V.

### Ion Transmission Efficiency Measurements

Measurements of ion transmission efficiency were carried out in a custom vacuum chamber containing a set of ion optics and a Faraday cup (FIG. 7A). The emission current was measured by a 2410 SourceMeter (Keithley Instruments), which also applied a high voltage to the tip via an Ag electrode. The leakage current through the BNC cable connecting the sourcemeter to the tip was measured and subtracted off of the measured emission current. The current striking the Faraday cup was measured using an SR570 current-preamplifier (Stanford Research Systems) connected to an NI PCIe-6251 DAQ card (National Instruments). The optics voltages were controlled using an 8-channel high voltage power supply (CAEN DT8033). A custom Labview program was used to control the voltage applied to the tip and record the emitted and transmitted currents.

### Magnetic Sector Measurements

A rudimentary magnetic sector mass spectrometer was constructed by adding a magnet and a Faraday plate to the vacuum chamber described above. The magnet consisted of a neodymium magnet with a yoke constructed from low-carbon magnetic iron (ASTM A848). The yoke concentrated the field in a flat circular region, 6 cm in diameter and 1 cm in height, which was directly downstream of the ion optics. The field strength within the flat circular region was measured to be B=0.54+/−0.02 T using a magnetometer. The Faraday plate used was a stainless steel disk, 4 cm in diameter, and 0.02 inch thick, connected directly to an electrical feedthrough by a steel wire. The Faraday plate was mounted at 45° relative to the Faraday cup, at the same distance from the center of the magnetic sector. The current emitted from the tip was measured using a 2410 Sourcemeter (Keithley), and the ion and droplet currents were each measured using a separate SR570 current preamplifier (Stanford Research Systems).

### Supplemental InformationSingle Amino Acid Ion Measurement Conditions

FIG. 6B presents mass spectra obtained from aqueous amino acids solutions. Table 2 displays the relevant experimental parameters for those data.

TABLE 2
Experimental conditions used to measure amino acid ions spectra in FIG. 6B

| | | Concentration | | K | Tip ID | Tip OD | P | Vₑ | Iₑ |
|---|---|---|---|---|---|---|---|---|---|
| Amino Acid | Mass | (mM) | pH | (S/m) | (nm) | (nm) | (torr) | (V) | (pA) |
| Arginine | 174 | 100 | 8.05 | 0.205 | 20 | 64 | 8 × 10⁻⁸ | 230 | 2 |
| Lysine | 146 | 100 | 5.75 | 0.486 | 20 | 64 | 5 × 10⁻⁸ | 260 | 1 |
| Histidine | 155 | 100 | 6.22 | 0.247 | 20 | 64 | 1.3 × 10⁻⁷ | 280 | 2 |
| Glycine | 75 | 100 | 4 | 1.96 × 10⁻² | 25 | 58 | 6 × 10⁻⁸ | 293 | 4 |
| Alanine | 89 | 100 | 4.01 | 1.94 × 10⁻² | 25 | 58 | 6 × 10⁻⁸ | 293 | 4 |
| Proline | 115 | 100 | 3.84 | 1.25 × 10⁻² | 25 | 58 | 6 × 10⁻⁸ | 293 | 4 |
| Valine | 117 | 100 | 4.08 | 1.75 × 10⁻² | 25 | 58 | 6 × 10⁻⁸ | 293 | 4 |
| Threonine | 119 | 100 | 3.94 | 1.40 × 10⁻² | 25 | 58 | 6 × 10⁻⁸ | 293 | 4 |
| Cysteine | 121 | 100 | 3.95 | 1.58 × 10⁻² | 25 | 58 | 6 × 10⁻⁸ | 293 | 4 |
| Leucine | 131 | 100 | 3.86 | 2.10 × 10⁻² | 25 | 58 | 6 × 10⁻⁸ | 293 | 5 |
| Phenylalanine | 165 | 100 | 4.1 | 1.08 × 10⁻² | 25 | 58 | 6 × 10⁻⁸ | 293\| | 4 |
| Serine | 105 | 100 | 4.08 | 1.56 × 10⁻² | 58 | 111 | 6 × 10⁻⁸ | 311 | 160 |
| Asparagine | 132 | 100 | 3.81 | 1.74 × 10⁻² | 58 | 111 | 1.4 × 10⁻⁷ | 380 | 240 |
| Methionine | 149 | 100 | 3.9 | 1.35 × 10⁻² | 58 | 111 | 6 × 10⁻⁸ | 311 | 160 |
| Tryptophan | 204 | 50 | 4 | 0.50 × 10⁻² | 58 | 111 | 6 × 10⁻⁸ | 311 | 160 |
| Glutamine | 146 | 100 | 3.39 | 5.58 × 10⁻² | 57 | 115 | 3 × 10⁻⁸ | 362 | 50 |

The spectra were measured in positive ion mode, for which it was necessary to lower the pH below the isoelectric point of the dissolved amino acid. This was done by the addition of acetic acid. The pH and the conductivity, K, of each solution are reported in Table 2. Ions were emitted directly into high vacuum from a nanopore ion source. The nanocapillaries had tip inner diameters in the range 20 nm-58 nm, and a single tip was frequently used to measure multiple amino acid solutions. Table 2 reports the inner and outer tip diameters of each nanocapillary used to obtain the data in FIG. 6B, and measurements taken from the same tip are indicated by the tip number. Table 2 also reports the time-averaged pressure P of the vacuum chamber, extraction voltage Vₑ, and emission current Iₑ, which were continuously monitored during the experiments.

Probability that an Emitted Ion Collides with a Gas Molecule

Amino acid ions were detected predominantly in unsolvated states in FIG. 6B. Collisions with gas molecules is the mechanism by which solvent molecules are separated from ions in conventional electrospray ionization. However, the instrument operated under high vacuum conditions where collisions with gas molecules were presumably rare. The kinetic theory of gases was used to calculate the probability that an emitted ion cluster would collide with at least one gas molecule. This addresses the question of whether the ions emerged from the solution unsolvated, or whether they emerged with a solvation shell which was knocked off by collisions with gas molecules on their way to the detector.

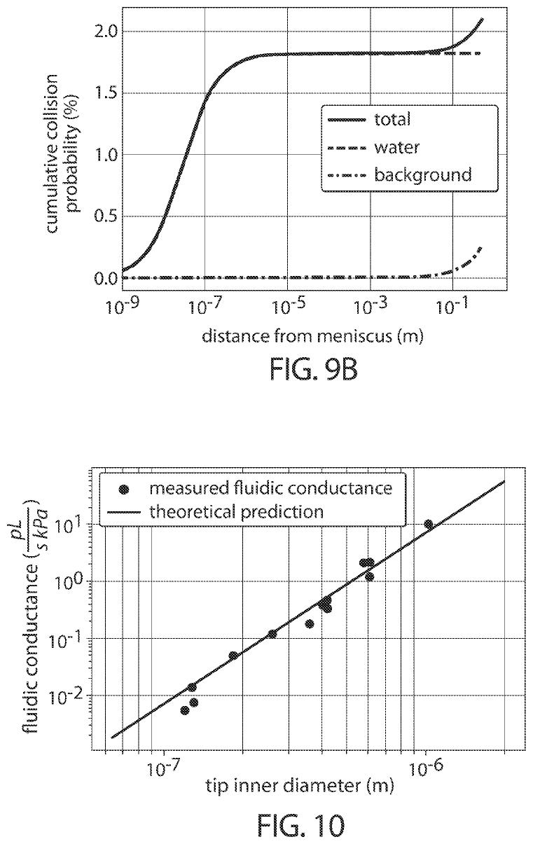

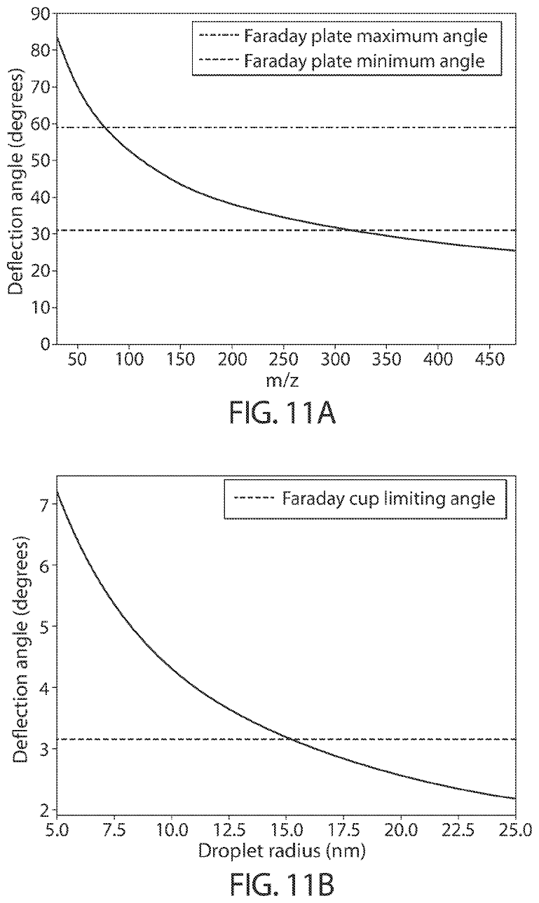

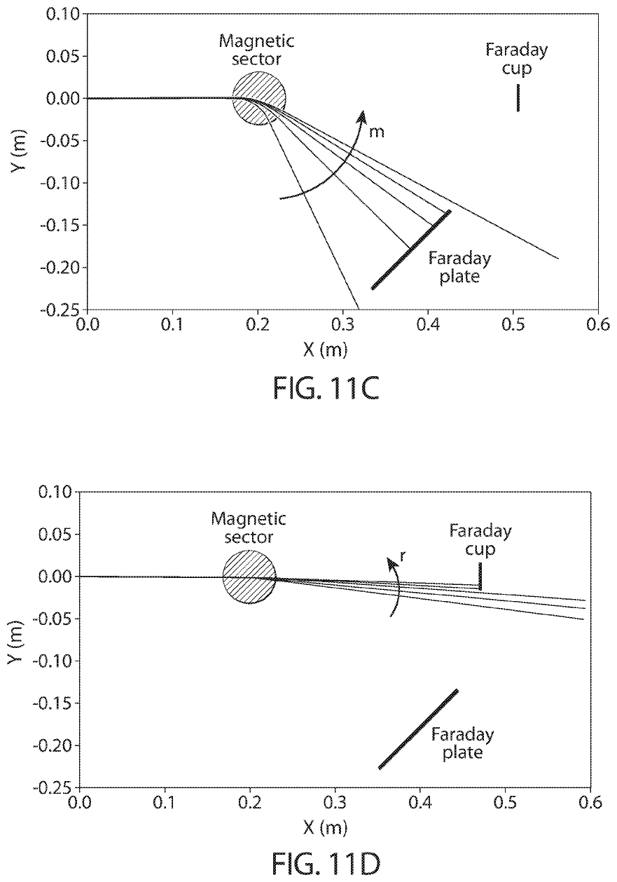

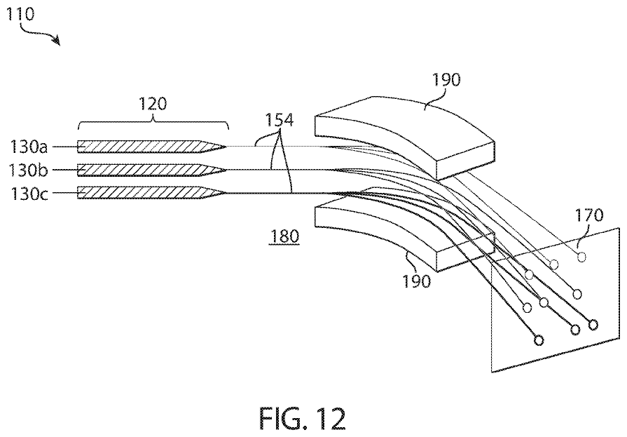

The distribution of gas within the vacuum chamber was taken to be the sum of two components, a uniform background of density ng and a distribution of water molecules nw that evaporated from the meniscus at the nanocapillary tip. The background gas pressure in this Example was typically around 7×10⁻⁸ torr (see Table 2). The mean free path of a water molecule in nitrogen gas at this pressure was over 1 km, suggesting that the evaporating water molecules traveled along ballistic trajectories away from the meniscus. The meniscus was modeled as a hemisphere and the evaporating water molecules as travelling radially outward, as illustrated in FIG. 9A. The density of water molecules decayed as n_{w}˜r⁻², where r is the distance from the center of the hemisphere. Although the rate at which water evaporated from a liquid meniscus into vacuum could not be experimentally well established, it was known that the flux of water molecules evaporating from the liquid surface cannot exceed the flux of incoming molecules at equilibrium. Thus, by substracting the incoming molecules, the highest possible density that the (outgoing) evaporating water molecules could achieve on the vacuum side of the liquid interface would be half the water vapor density at equilibrium. The maximum probability that an emitted ion could collide with a gas molecule was found by assuming the density of water vapor on the vacuum side of the meniscus is

$$\frac{{p}_{0}}{2 {k}_{B} T} ,$$

where p₀=17.5 torr is the approximate equilibrium vapor pressure of water at 20° C. and k_{B}T is the thermal energy.

The cumulative probability that an ion cluster undergoes at least one collision before reaching r, Cₚ(r) was also determined. To compute Cₚ(r), it is easier to consider Pₛ(r)=1−Cₚ(r), the probability that an ion survives to a distance r without undergoing a collision. Pₛ(r) is related to Pₛ(r−dr) by the probability of colliding with a gas molecule within the interval r−dr→r, represented by the stochastic process P_{c}(r−dr→r),

$${P}_{s} ( r ) = {P}_{s} ( r - dr ) ( 1 - {P}_{c} ( r - dr \to r ) ) . \qquad ( 5 )$$

In the limit of dilute gasses and infinitesimal displacements,

$${P}_{c} ( r - dr \to r ) = {σ}_{w} {{n}_{w , 0} ( \frac{{r}_{0}}{r} )}^{2} dr + {σ}_{b} {n}_{b} dr , \qquad ( 6 )$$

where σ_{w} is the cross section for a collision between an ion cluster and a water molecule, σ_{b} is the cross section for an ion and a background gas molecule, n_{w,0} is the number density of water on the vacuum side of the meniscus, r₀ is the radius of the meniscus, and n_{b} is the number density of background gas molecules. Combining Eqs. 5 and 6 and rearranging gave rise to a differential equation for Pₛ

$$\frac{{P}_{s} ( r ) - {P}_{s} ( r - dr )}{{P}_{s} ( r - dr )} = \frac{{dP}_{s} ( r )}{{P}_{s} ( r )} = - {σ}_{w} {{n}_{w , 0} ( \frac{{r}_{0}}{r} )}^{2} dr - {σ}_{b} {n}_{b} dr . \qquad ( 7 )$$

Integrating Eq. 7 from r₀ to r, applying the boundary condition Pₛ(r₀)=1, and solving for Pₛ(r) gave

$${P}_{s} ( r ) = exp [ - {σ}_{w} {n}_{0} ( \frac{1}{{r}_{0}} - \frac{1}{r} ) - {σ}_{b} {n}_{b} ( r - {r}_{0} ) ] . \qquad ( 8 )$$

Finally, the cumulative probability that an ion experiences at least one collision before reaching r is given by

$${C}_{P} ( r ) = 1 - {P}_{s} ( r ) . \qquad ( 9 )$$

FIG. 9B plots Cₚ(r) in this Example. The cross sections σ_{w} and σ_{b} are given respectively by π(aᵢ+a_{w})² and π(aᵢ+a_{b})², where a_{w}=1.325 Å is the kinetic radius of water, a_{b}=1.82 Å is the kinetic radius of the background gas molecules, and aᵢ=7 Å is the approximate radius of an amino acid with a full solvation shell of water. n_{b}=2.25×10¹⁵ m⁻³ and n_{w,0}=6.44×10²³ were taken to be the number densities of background gas and water molecules on the vacuum side of the meniscus, respectively. The meniscus radius is r₀=30 nm. Cₚ(r) raised rapidly over the first 100 nm before saturating at 1.8%. Cₚ(r) increased slowly over centimeter-scale distances due to the finite density of background gas molecules; Cₚ(r) reached 2.1% over the 50 cm distance from the ion source to the detector.

These results showed that, in contrast to conventional electrospray, collisions between gas particles and ion clusters emitted from the nanopore ion source were rare. The majority of ions followed collision-less trajectories from the source to the detector because the instrument operated under high vacuum conditions. The lack of collisions implies that ions were emitted in the same state as when they were detected, leading to the conclusion that the nanopore ion source may be capable of emitting predominantly unsolvated amino acid ions.

### The Nanopore Ion Source Operates Below the Minimum Stable Flow Rate for Cone-Jet Electrospray

A minimum flow rate Qₑ may exist below which a stable cone-jet electrospray cannot exist. Expressions for Qₑ valid for cone-jets of polar liquids with either high or low Reynolds number Rₑ are presented herein If a cone-jet of aqueous amino acid solution were to form, Rₑ>1 would be expected, so the appropriate expression for Qₑ would have been

$${Q}_{ϵ} = \frac{{ϵ}_{0} ϵγ}{ρ K} , \qquad ( 10 )$$

where ϵ₀ and ϵ are the vacuum permittivity and relative permittivity respectively, γ is the surface tension, ρ is the density, and K is the conductivity. Using Eq. 10, it was predicted that Qₑ=5×10⁻¹³ m³/s, or 30 nL/min for measurements of amino acid solutions, given ϵ=80, γ=0.073 N/m, ρ=1000 kg/m³, and K=0.1 S/m.

The expected flow rate through a nanopore ion source is at least three orders of magnitude below Qₑ. The flow rate through nanocapillaries was measured by blowing water droplets into silicone grease at a fixed applied pressure and measuring the growth rate of the droplet. A nanocapillary was initially filled with water and its back end was connected to a nitrogen cylinder through a pressure regulator. The tip of the capillary was submerged in a dish of silicone grease under an optical microscope. The back pressure was slowly increased from the nitrogen cylinder until it could overcome the Laplace pressure of the water-grease interface at the nanocapillary tip and begin to inflate a droplet. Videos of the droplet growing under a fixed back pressure were recorded at an image rate of 10 Hz. FIG. 10 plots the fluidic conductance of 14 nanocapillaries as a function of the inner diameters of their tips. The flow rate through the smallest tip measured in this way, having an inner diameter of 120 nm, would be 30 pL/min at 1 atm of applied pressure. Hence, even a larger tip with a diameter of 100 nm would only permit a flow rate that is three orders of magnitude below the minimum flow rate needed to sustain a stable cone-jet.

FIG. 10 also plots the theoretical flow rate through a nanocapillary. That calculation takes the nanocapillary geometry to be a semi-infinite truncated cone with a tip inner diameter r₀. The apex half angle θ was used as a fitting parameter. The Poiseuille flow through that cone obtains the fluidic conductance

$$\frac{Q}{Δ P} = \frac{3 π tan θ {r}_{0}^{3}}{8 μ} , \qquad ( 11 )$$

where ΔP is the pressure drop across the capillary, and μ is the viscosity. A least squares fit of Eq. 11 to the data in FIG. 10 gave rise to θ=2.5°. The data in FIG. 7A was obtained by multiplying the fluidic conductances in FIG. 10 by an applied pressure of 1 atm and the minimum flow rate Qₑ calculated from Eq. 10 is also shown.

### Simulations of Ion and Droplet Trajectories Through Magnetic Sector

Simulations were carried out using custom Python code to determine the m/z range of particles expected to strike the Faraday cup and Faraday plate detectors used in the magnetic sector experiments. Particles were assigned a m/z ratio and given an initial kinetic energy of qV_{T}, where q is the charge on the particle and V_{T} is the tip voltage. The trajectories of the particles through the magnetic sector were then calculated numerically by solving the Lorentz force law using a fourth-order Runge-Kutta scheme. The magnetic field was assumed to be 0.54 T in the z-direction within the bounds of the magnetic sector (a circle with a diameter of 6 cm), and 0 everywhere else. Given the geometry of the instrument and placement of the detectors relative to the magnetic sector, the minimum/maximum deflection angles needed for particles to strike the two detectors can be calculated. In order to strike the Faraday cup and contribute to the measured droplet current, I_{Drop}, a particle may be deflected by between 0° and 3.15°, and for the Faraday plate (I_{Ion}), a particle may be deflected by between 30.96° and 59.04°. It was found that particles with 75<m/z<315 will strike the Faraday plate, corresponding to singly charged sodium ions with between 3 and 16 additional water molecules attached. It was also found that particles with m/z>37000 would strike the Faraday cup, this corresponds to a water droplet charged to the Rayleigh limit with a radius of >15 nm. It should be noted that droplets charged below the Rayleigh limit will be deflected less.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of.” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1-25. (canceled)

26. A method comprising:
arranging a protein into a substantially linear configuration in a nanotip;
fragmenting the protein into amino acids by applying laser light to the protein;
emitting the amino acids from the nanotip; and
detecting the amino acids emitted from the nanotip.

27-30. (canceled)

31. The method of claim 26, wherein the method has a mean ion transmission efficiency of at least 85%.

32-33. (canceled)

34. The method of claim 26, wherein the nanotip comprises an opening having a cross-sectional dimension of less than 100 nm.

35. (canceled)

36. The method of claim 26, wherein the nanotip comprises an opening having a cross-sectional dimension of less than or equal to 60 nm.

37. The method of claim 26, wherein the nanotip comprises an opening having a cross-sectional dimension of less than or equal to 25 nm.

38. (canceled)

39. The method of claim 26, wherein the nanotip comprises an opening having a cross-sectional dimension of greater than or equal to 1 nm.

40. The method of claim 26, wherein the emitted amino acids are in the form of bare ions and/or ion clusters.

41. The method of claim 26, wherein at least 80% of the emitted amino acids are in the form of bare ions.

42. (canceled)

43. The method of claim 26, wherein the emitted amino acids are emitted in a sequential fashion.

44. The method of claim 26, wherein detecting the amino acids comprises detecting the amino acids in an order the amino acids are emitted from the nanotip.

45. The method of claim 26, further comprising determining a sequence of the protein by determining the emitted amino acids with the detectors.

46-61. (canceled)

62. A mass spectrometer, comprising:
an ion source comprising a capillary;
a light source directed towards the ion source, wherein the light source is able to produce light at a wavelength of greater than or equal to 150 nm and less than or equal to 213 nm;
a magnetic mass filter downstream of the ion source;
an array of detectors downstream of the magnetic mass filter.

63. The mass spectrometer of claim 62, wherein the light source is able to produce light at a wavelength of 193 nm+/−5 nm.

64. The mass spectrometer of claim 62, wherein the light source is a laser.

65. The mass spectrometer of claim 62, wherein the ion source comprises an electrode proximate the capillary.

66. The mass spectrometer of claim 62, wherein the capillary comprises a body portion and a tip portion fluidically connected to the body portion.

67-73. (canceled)

74. The mass spectrometer of of claim 62, further comprising one or more additional ion sources adjacent the ion source.

75. (canceled)

76. The mass spectrometer of claim 74, wherein at least one of the one or more additional ion sources comprises a capillary.

77. (canceled)

78. The mass spectrometer of claim 62, wherein the array of detectors downstream the magnetic mass filter is arranged in a 2-dimensional array.

79-83. (canceled)

84. A method of sequencing a protein, comprising:
passing a fluid comprising a protein into a capillary defining an opening;
applying light at a wavelength of greater than or equal to 150 nm and less than or equal to 213 nm to the protein proximate the opening to produce fragments;
passing the fragments directly into an environment having a pressure of no more than 100 mPa;
passing the fragments through a magnetic mass filter;
directing the fragments to an array of detectors; and
determining a sequence of the protein by determining the fragments with the array of detectors.

85-87. (canceled)
